(19) United States
(12) Patent Application Publication
(10) Pub. No.: US 2023/0138274 A1
(43) Pub. Date: May 4, 2023

(54) Body Fluids Sampling Device and Method of using the Same

(71) Applicant: Preci Health SA, Neuchâtel (CH)

(72) Inventors: Lucien Vouillamoz, Feusisberg (CH); Alain JACCARD, Ste-Croix (CH); Michel Gilbert BRUEHWILER, Newton, MA (US); Michel ORSINGER, Bursinel (CH); Craig COOK, Neuchåtel (CH); Philippe MARGAIRAZ, La Chaux-de-Fonds (CH); Luc MAFFLI, Fribourg (CH); Toraif BORK, Enges (CH); Bartosz KOREC, Palm Harbor, FL (US); Philip D'Almada REMEDIOS, Palm Harbor, FL (US); Robert Sean HÄGEN, Ozona, FL (US)

(73) Assignee: Preci Health SA, Neuchâtel (CH)

(21) Appl. No.: 17/915,497

(22) PCT Filed: Mar. 31, 2021
(86) PCT No.: PCT/IB2021/000187

§ 371 Date: Sep. 29, 2022

Related U.S. Application Data
(60) Provisional application No. 63/002,581, filed on Mar. 31, 2020.
(60) Provisional application No. 63/006,337, filed on Apr. 7, 2020.
(60) Provisional application No. 63/011,010, filed on Apr. 16, 2020.
(60) Provisional application No. 63/025,692, filed on May 15, 2020.
(60) Provisional application No. 63/069,112, filed on Aug. 23, 2020.
(60) Provisional application No. 63/114,162, filed on Nov. 16, 2020.
(60) Provisional application No. 63/142,756, filed on Jan. 28, 2021.
(60) Provisional application No. 63/150,113, filed on Feb. 17, 2021.
(60) Provisional application No. 63/153,088, filed on Feb. 24, 2021.

(51) Int. Cl. A61B 5/15; A61B 5/1171; A61B 5/151; G06T 7/00; A61M 5/31; G09B 19/00; G16H 40/67; G06Q 10/0832

(52) U.S. Cl. CPC A61B 5/150801; A61B 5/150458; A61B 5/150786; A61B 5/150343; A61B 5/150854; A61B 5/1176; A61B 5/15109; G06T 7/0012; A61M 5/31; G09B 19/003; G16H 40/67; G06Q 10/0832; G06T 2207/10016; G06T 2207/30004; A61M 2205/3569; A61M 2205/3553; A61M 2202/30; A61M 2205/3368; A61M 2205/505; A61M 2205/18

(57) ABSTRACT

A fluid sampling device and a method is provided for collecting body fluid samples such as blood without the intervention of medically trained personnel. The body fluid sampling device having a sample containment chamber made of a material having a thermal inertia permitting the maintenance of sample temperature over a known period of time. Optionally an isolating cover or sleeve may slide in place by a mechanism triggered by thermal contraction of an element after the device has reached a sufficiently low temperature in a patients refrigerator. Associated methods are provided to ensure hermetic transport of the collected body fluid sample to an analysis lab. Also disclosed is a device and app combination for drug or vaccine injection, the app including means to allow for verification of the patient's ID and/or the particular device used. The device my be equipped with geo-localization and long-range communication capabilities.

### CROSS REFERENCE TO RELATED APPLICATIONS

This application claims the benefit of U.S. Provisional Application No. 63/002,581, filed Mar. 31, 2020, entitled BODY FLUIDS SAMPLING DEVICE AND METHOD OF USING THE SAME, No. 63/006,337, filed Apr. 7, 2020, entitled BODY FLUIDS SAMPLING DEVICE AND METHOD OF USING THE SAME, No. 63/025,692, filed May 15, 2020, entitled BODY FLUIDS SAMPLING DEVICE AND METHOD OF USING THE SAME, No. 63/011,010, filed Apr. 16, 2020, entitled BODY FLUIDS SAMPLING METHOD WITH AUTOMATIC USER IDENTIFICATION, No. 63/069,112, filed Aug. 23, 2020, entitled CAPILLARY BLOOD SAMPLING DEVICE AND METHOD OF USING THE SAME, No. 63/114,162, filed Nov. 16, 2020, entitled SELF-MADE MEDICAL PROCESS VERIFICATION SYSTEM, No. 63/142,756, filed Jan. 28, 2021, entitled CAPILLARY BLOOD SAMPLING SYSTEM INCLUDING USER/PATIENT AUTHENTICATION, No. 63/150,113, filed Feb. 17, 2021, entitled CAPILLARY BLOOD SAMPLING DEVICE, and No. 63/153,088, filed Feb. 24, 2021, entitled VACCINE AND/OR DRUG INJECTION AND ADMINISTRATION DEVICE.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the respective Patent and Trademark Office patent file or records, but otherwise reserves all copyright rights whatsoever. Further, no references to third party patents or articles made herein is to be construed as an admission that the present invention is not entitled to antedate such material by virtue of prior invention.

### BACKGROUND OF THE INVENTION

This invention relates to devices allowing for the sampling of body fluids and their transport to an analysis lab without the intervention of medical personnel. In cases of war or large epidemics, it may become necessary to analyze the body fluids, for example the blood, of a substantial part of a given population, potentially the whole population of a neighborhood, a block or group of blocks, a suburb, a city, a whole country or a continent. In such situations, it may not be possible for medical personnel to personally attend to the sampling of the body fluids of every patient for several reasons such as confinement obligation, risk of contamination, unsafe areas, travelling distances, lack of transport infrastructures, and/or lack of personnel. What is needed therefore is a fluid sampling device adapted to be used by any individual on himself without rigorous medical training, and a method to ensure the authentication of the user/patient and the association between the authenticated user/patient and the body fluid sample, as well as the intact, safe and sanitary transport of the body fluid sample to an analysis lab.

### SUMMARY OF THE INVENTION

A fluid sampling device and methods are provided consisting of collecting body fluid samples such as blood without the intervention of medically trained personnel. The body fluid sampling device optionally advantageously includes an insulating cover or sleeve adapted to slide over the sample container so as to extend the possible transport times. In one embodiment, the isolating cover or sleeve is manually set into place by the user via a tab actuated by the user according to written instructions provided with the device, or automatically slid into place by a second mechanism optionally triggered by thermal contraction of an element after the device has reached a sufficiently low temperature in the refrigerator. The body fluid sampling device optionally is equipped with a unique identification code, and optionally carries an electronically readable identification tag such as an RFID readable tag. Depending on the circumstances, the sampling device is optionally equipped with geo-localization and long-range communication capabilities so as to be collectable without any further action from the user after the sampling process has been executed. Advantageously, the body fluid sampling device according the invention is optionally configured to use standard analysis tubes as well-known in the industry, so that the tubes' content can be analyzed on standard automated analysis equipment. Associated methods are provided for authenticating the user/patient, associating with or ensuring the correspondence between the authenticated user/patient and the sample container, optionally with the sampling device, and using such sampling device to collect a sample of body fluid and to ensure the hermetic, sanitary, intact and safe transport of the collected body fluid sample to an analysis lab.

### BRIEF DESCRIPTION OF THE DRAWINGS

FIG. 1A is a flowchart of a first embodiment of the method of the invention.

FIG. 1B is a flowchart of a second embodiment of the method of the invention.

FIG. 1C is a flowchart of a third embodiment of the method of the invention.

FIG. 1D is a flowchart of a fourth embodiment of the method of the invention.

FIG. 2A is a perspective view of the internal mechanism of a sampling device, showing a first embodiment of the cutting blade of the invention before use.

FIG. 2B is a perspective view of the internal mechanism of a sampling device according to the invention while in use.

FIG. 2C is a perspective view of the internal mechanism of a sampling device according to the invention after use.

FIG. 3A is a side view of the internal mechanism of a sampling device according to the invention before use.

FIG. 3B is a perspective view showing the internal mechanism of a sampling device according to the invention while in use.

FIG. 3C is a side view of the internal mechanism of a sampling device according to the invention while in use.

FIG. 3D is the side view of the internal mechanism of a sampling device according to the invention after use.

FIG. 4 is a lower perspective view of the contact surface of a sampling device according to the invention with the user's skin.

FIG. 5 is a perspective view of the internal fluidic channel of a sampling device according to the invention.

FIG. 6 is a plan view of a thermal indicator panel according to the invention.

FIGS. 7A-7B are partial cross-section views of the internal elements of a sampling device according to the invention, showing the extraction of the body fluid sample.

FIGS. 8A-8B are cross sectional views showing two stages of movement of the body fluid sampling device of the invention, namely, the step before use (FIG. 8A), and then the step after sampling (FIG. 8B).

FIG. 9 is a perspective view of another embodiment of the body fluid sampling device of the invention for the collection of capillary blood from the ear lobe.

FIGS. 10A-10B are schematic views of the body fluid sampling device for the collection of capillary blood from the ear lobe.

FIG. 11 is a flow chart of a fifth embodiment of the method for sampling body fluids of the invention, ensuring the identification of the user-patient.

FIG. 12, is a side view of an embodiment of capillary blood sampling device according the invention which has a main structure containing the suction interface adapted to be attached to the skin of the user/patient.

FIG. 13 is a localized, partial, cross-sectional view of the embodiment of capillary blood sampling device of FIG. 12 showing a second embodiment of the cutting blade of the invention, the device in contact with the user/patient through its suction interface.

FIGS. 14A to 14I are partial, cross-sectional views showing the functioning of the device of the invention for use with a standard sample container.

FIG. 15A is a side view of the second embodiment of a cutting blade, or lancet, for use in the invention, in a position ready for release.

FIG. 15B is side view of the second embodiment of the cutting blade for use in the invention, in a position ready half-way extended.

FIG. 15C is a top view of the second embodiment of the cutting blade fully extended.

FIG. 16A is a perspective view of a third embodiment of the cutting blade of the invention, made from a single piece of metal.

FIG. 16B is a side view of the third embodiment of the cutting blade of the invention.

FIG. 16C is a front view of the third embodiment of the cutting blade of the invention.

FIG. 16D is a top view of the third embodiment of the cutting blade of the invention.

FIG. 16E is a side view of the third embodiment of the cutting blade of the invention, half extended.

FIG. 16F is a side view of the third embodiment of the cutting blade of the invention, fully extended.

FIG. 17 is a multiturn torsion spring that may be used in the elastic zone in a particular embodiment of the invention.

FIG. 18A is an upper, perspective view of another embodiment of the blood sampling device of the invention.

FIG. 18B is a lower, perspective view of another embodiment of the blood sampling device of the invention.

FIG. 19 is a perspective view of an injection device of the invention.

FIGS. 20A to 20P are schematic views illustrating the steps of a sixth embodiment of the method of using the system of the invention.

FIGS. 21A to 21D are schematic views with more detail about the specific features of the adhesive integrated dressing included in the capillary blood sampling device.

FIG. 22A is a perspective view of a fourth embodiment of the cutting blade of a device of the invention is made of a single part.

FIG. 22B is a side, four-position, shutter view of the fourth embodiment of the cutting blade of a device of the invention.

FIG. 22C is a side, four-position, shutter view of the fourth embodiment of the cutting blade of a device of the invention showing the retraction thereof.

FIG. 23A is a perspective view of a fifth embodiment of the cutting blade of the invention.

FIG. 23B is a front view of the fifth embodiment of the cutting blade of the invention.

FIG. 24A in side view of a sixth embodiment the cutting blade of a device of the invention, prior to entry into the epidermal layer of the patient's body.

FIG. 24B in side view of the sixth embodiment the cutting blade of a device of the invention half way through its cycle of motion.

FIG. 24C in side view of the sixth embodiment the cutting blade of a device of the invention at the end of its cycle of motion, having returned to its initial position.

FIG. 25A is a perspective view of a seventh embodiment the cutting blade of a device of the invention showing a standard scalpel blade.

FIG. 25B is a side view of the seventh embodiment the cutting blade of a device of the invention, prior to entry into the epidermal layer.

FIG. 25C is a side view of the seventh embodiment of the cutting blade of a device of the invention after entry into the epidermal layer, as it is about to be removed from said layer.

FIG. 25D is a side view of the epidermal layer showing the cut made by the seventh embodiment of the cutting blade.

FIG. 26A to FIG. 26C show examples of visible features that can be integrated in a device of the invention to be easily recognized by the human observer, or easily identified by a real-time image analysis software.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’, ‘back’, ‘top’ and ‘bottom’, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### DETAILED DESCRIPTION OF THE PREFERRED EMBODIMENT

The following description is not intended to limit the scope of the invention in any way as they are exemplary in nature, serving to describe the best mode of the invention known the inventors as of the filing date hereof. Consequently, changes may be made in the arrangement and/or function of any of the elements described in the exemplary embodiments disclosed herein without departing from the spirit and scope of the invention.

The body fluid sampling device according the invention may take on different sizes and shapes, depending on the fluid(s) and size of the sample to be collected, and on the area of the body where the sample is to be collected. The sample may be collected on the skin or on a mucosa, but the sample may have to be collected through the skin/mucosa, in such case the sampler may be equipped with a cutting blade to open the skin/mucosa and release the body fluid to be sampled. A reactant may be added by the user on the surface of the skin/mucosa to facilitate the conservation of the sample or to enable specific analyses. Optionally, the thermal inertia of the body fluid sampling device is adapted to the above parameters and to the transport constraints, so that the storage temperature of the body fluid sample is kept optimal from the user's fridge to the fridge of the analysis lab with sufficient margin. The transport of the collected sample from the user's location to the analysis lab may be done by the user himself, by postal services, or by a dedicated logistics organization using human transporters or autonomous or remotely controlled unmanned vehicles. Depending on the circumstances, the sampling device may be equipped with geo-localization and long-range communication capabilities so as to be collectable without any further action from the user after the sampling process has been executed.

The authentication of the patient and ensuring the correspondence between the authenticated user/patient and the sample, optionally with the sampling device, is optionally accomplished via differing levels of antitampering measures, as the stakes may be very high for the patient, for example, the result may determine whether the patient is placed in quarantine or released from quarantine, authorized to travel, authorized to work, it may serve as confirmation of the efficiency of a treatment, and/or the releasing of payment for a treatment, etc. Such anti-tampering measures may increase to include filming the entire body fluid sampling process from opening the box containing the sampling device to sealing the box to be mailed to the sample analysis organization, optionally encrypting, and storing the movie for later use, for example to be used as a proof in court in case of legal proceeds.

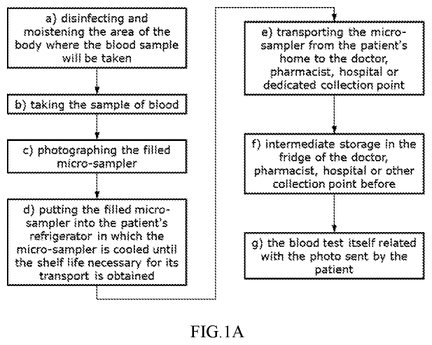

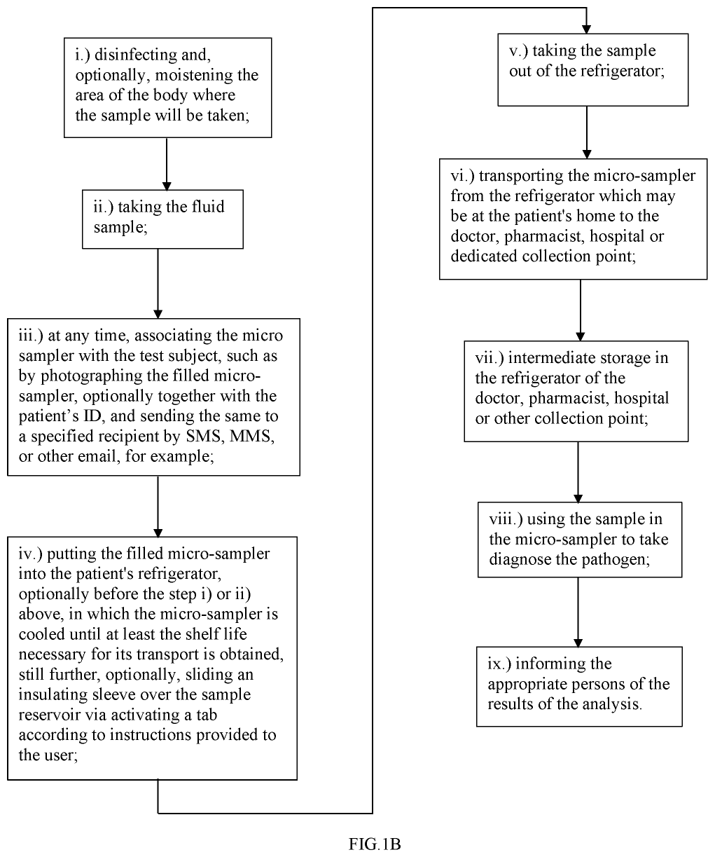

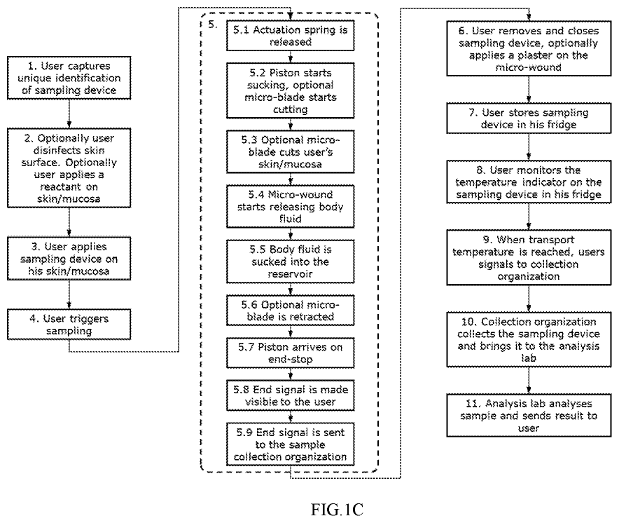

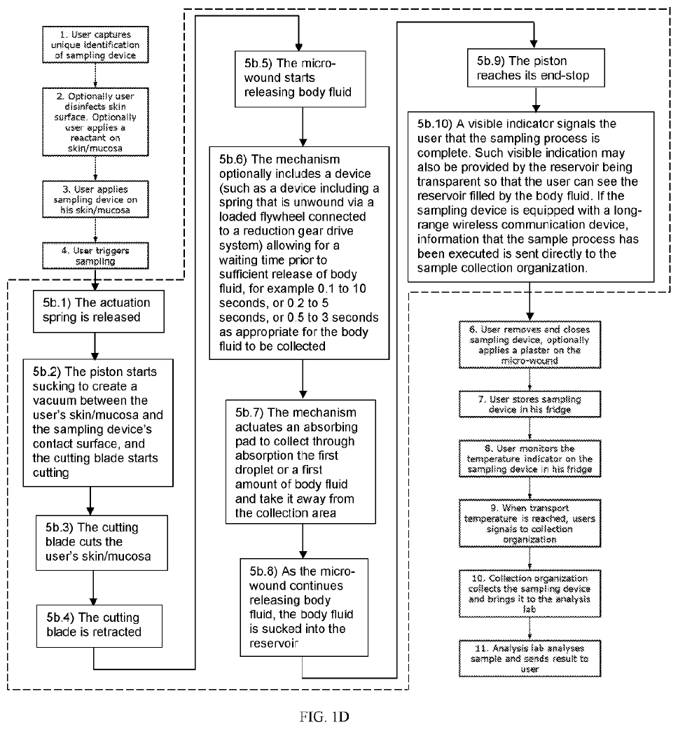

Referring now to FIG. 1A, a first embodiment of the method according to the invention includes the steps of:
a) disinfecting and moistening the area of the body where the blood sample will be taken;
b) taking the sample of blood;
c) photographing the filled micro-sampler and optionally the patient's ID;
d) putting the filled micro-sampler into the patient's refrigerator, optionally before the first step a) orb) above, in which the micro-sampler is cooled until the shelf life necessary for its transport is obtained, still further, optionally, sliding an insulating sleeve over the sample reservoir via activating a tab according to instructions provided to the user;
e) transporting the micro-sampler from the patient's home to the doctor, pharmacist, hospital or dedicated collection point;
f) intermediate storage, optionally in the refrigerator of the doctor, pharmacist, hospital or other collection point before; and
g) the blood test itself related with the photo sent by the patient.

Referring now to FIG. 1B, a second embodiment of the method according to the invention includes the steps of:
i. disinfecting and optionally moistening the area of the body where the sample will be taken;
ii. taking the fluid sample;
iii. at any time, associating the micro sampler with the test subject, such as by photographing the filled micro-sampler, optionally together with the patient's ID, and sending the same to a specified recipient by SMS, MIMS, or other email, for example;
iv. storing the filled micro-sampler optionally into the patient's refrigerator, optionally before the step i) or ii) above, in which the micro-sampler is cooled until at least the shelf life necessary for its transport is obtained, still further, optionally, sliding an insulating sleeve over the sample reservoir via activating a tab according to instructions provided to the user;
v. taking the sample out of the refrigerator;
vi. transporting the micro-sampler optionally from the refrigerator which may be at the patient's home to the doctor, pharmacist, hospital or dedicated collection point;
vii. intermediate storage optionally in the refrigerator of the doctor, pharmacist, hospital or other collection point;
viii. using the sample in the micro-sampler to take diagnose the pathogen; and
ix. informing the appropriate persons of the results of the analysis.

Referring now to FIG. 1C, a third embodiment of the method of the invention comprises the steps:
Step 1: The user captures the unique identification of his sampling device, with a dedication software application (app) (connected to the Internet or to the CLOUD™) on his smartphone capturing a QR code on his sampling device, by collecting an identification tag attached on the device when delivered, or any other appropriate means. Optionally the user associates the sampling device with the patient, for example by photographing the device with its identification visible together with the patient's ID document, or together with the patient's face.
Step 2: Optionally the user disinfects the skin surface, providing disinfection but optionally also moisturizing the surface, enabling better suction. Optionally the user applies a reactant on his skin/mucosa, such reactant to mix with the body sample to allow/improve the body sample conservation and/or to enable/improve analyses.
Step 3: The user applies the sampling device on his skin/mucosa at the appropriate area.
Step 4: The user triggers the sampling process, optionally after having cooled the device according to the previously described methods,
Step 5: The sampling process is executed automatically by the sampling device, in the following sub-steps:
Step 5.1: The actuation spring is released.
Step 5.2: The piston starts sucking to create a vacuum between the user's skin/mucosa and the sampling device's contact surface, and the optional cutting blade starts cutting.
   Step 5.3: The optional cutting blade cuts the user's skin/mucosa.
   Step 5.4: The micro-wound starts releasing body fluid.
   Step 5.5: Body fluid is sucked into the reservoir.
   Step 5.6: The optional cutting blade is retracted.
   Step 5.7: The piston reaches its end-stop.
   Step 5.8: A visible indicator signals the user that the sampling process is complete. Such visible indication may also be provided by the reservoir being transparent so that the user can see the reservoir filled by the body fluid.
   Step 5.9: If the sampling device is equipped with a long-range wireless communication device, information that the sample process has been executed is sent directly to the sample collection organization.
Step 6: The user detaches the sampling device from his skin/mucosa and closes it so as to protect the integrity of the body fluid sample. The user optionally applies a plaster on the micro-wound, the micro-wound closes itself naturally. Optionally, the user associates the sampling device with the patient, for example by photographing the device with its identification visible together with the patient's ID document, or together with the patient's face.
Step 7: Optionally before the sample is taken, the user stores the sampling device in his fridge.
Step 8: Optionally before the sample is taken, the user follows instructions to bring the temperature of the sampling device to appropriate temperature, optionally monitoring the evolution of the temperature of the sampling device by observing the temperature display. If the sampling device is equipped with a temperature sensor and with a long-range wireless communication device, information on the sampling device temperature is sent directly to the sample collection organization.
Step 9: When the instructions are followed, e.g. transport temperature is reached, the user optionally informs the sample collection organization that the sample is ready to be picked up or has the device delivered to the appropriate collection point. If the sampling device is equipped with a long-range wireless communication device, information about the sampling device readiness to be picked up may be sent directly to the sample collection organization.
Step 10: The sample collection organization collects the sampling device and brings it to the analysis lab.
Step 11: The analysis lab runs the sample analysis and informs the user and/or the appropriate authorities of the result by any communication means such as through a dedication software application (app) (connected to the Internet or the CLOUD™) in the user's smartphone or by any other appropriate communication means.

Capturing the identification of the sampling device (step 1) may also be done between steps 5 and 6 (after having run the sampling process)

Referring now to FIG. 1D, a fourth embodiment of the method of the invention has all the steps of the third embodiment of the method of the invention but instead of Step 5, a modified Step 5b as follows:
Step 5b: The sampling process is executed automatically by the sampling device, in the following sub-steps:
   Step 5b.1: The actuation spring is released.
   Step 5b.2: The piston starts sucking to create a vacuum between the user's skin/mucosa and the sampling device's contact surface, and the cutting blade starts cutting.
   Step 5b.3: The cutting blade cuts the user's skin/mucosa.
   Step 5b.4: The cutting blade is retracted.
   Step 5b.5: The micro-wound starts releasing body fluid.
   Step 5b.6: The mechanism optionally includes a device (such as a device including a spring that is unwound via a loaded flywheel connected to a reduction gear drive system) allowing for a waiting time prior to sufficient release of body fluid, for example 0.1 to 10 seconds, or 0.2 to 5 seconds, or 0.5 to 3 seconds as appropriate for the body fluid to be collected.
   Step 5b.7: The mechanism actuates an absorbing pad to collect through absorption the first droplet or a first amount of body fluid and take it away from the collection area.
   Step 5b.8: As the micro-wound continues releasing body fluid, the body fluid is sucked into the reservoir.
   Step 5b.9: The piston reaches its end-stop.
   Step 5b.10: A visible indicator signals the user that the sampling process is complete. Such visible indication may also be provided by the reservoir being transparent so that the user can see the reservoir filled by the body fluid. If the sampling device is equipped with a long-range wireless communication device, information that the sample process has been executed is sent directly to the sample collection organization.

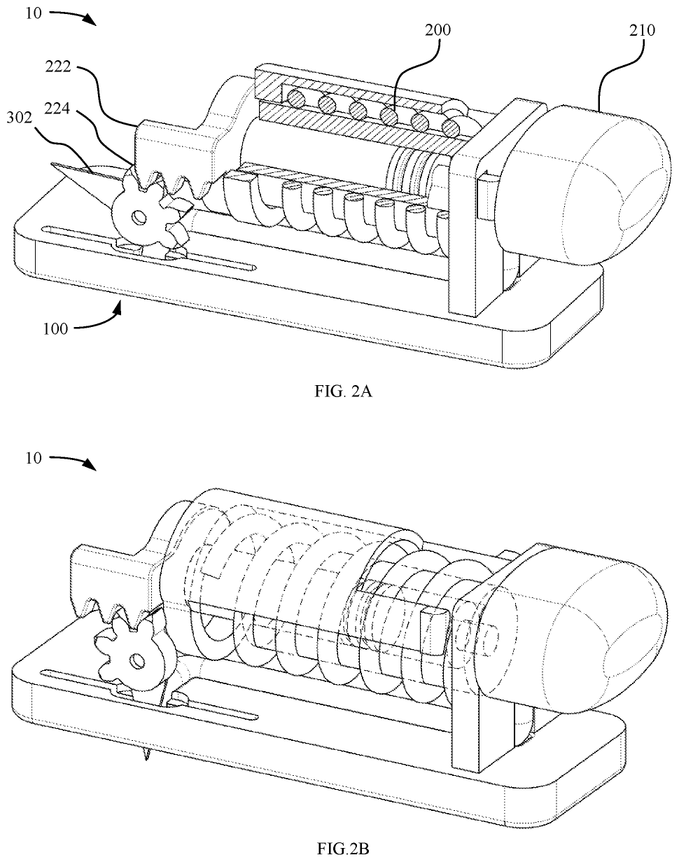

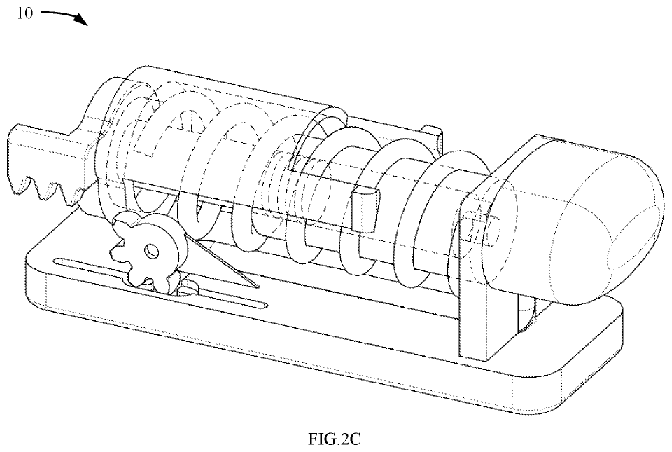

Referring now to FIGS. 2A-2C, the internal mechanism 10 of the body fluid sampling device contains an energy source 200 that is sufficient to ensure the execution of the entire sampling process, for example a spring. The device includes a rigid reservoir 502 closed by a plunger 504. The mechanism 10 connects the energy source 200 to the plunger 504 via a piston 242 so that the plunger 504 can be retracted in order to create vacuum and suck the body fluid sample. Depending on the type of body fluid to be collected and the skin/mucosa protecting it, optionally the mechanism 10 also connects the energy source 200 to a first embodiment of a cutting blade 302 that is able to cut the user's skin/mucosa in order to release the body fluid sample to be collected. The mechanism 10 includes a trigger 210 that can be activated by the user to launch the sampling process. The mechanism 10 may include gears, levers, cams, snaps, racks, pinions, springs, and any other mechanic components 220, 222, 224 the selection of a suitable assembly of which is within the skill of a person of ordinary skill in the micromechanics industry so as to ensure the execution of the entire sampling process without any other action from the user after the trigger 210 has been released.

Referring now to FIG. 2B, the sampling device 10 (also devices 1100, 2010, 2210, 4100, 3100) has a unique identification that can take many forms: a QR code that may be read by the user's smartphone or a number that the user may write down for reference, etc. The sampling device 10 (and 1100, 2010, 2210, 4100, 3100 as the case may be) optionally contains a long-range wireless communication device in order to be geo-localized and to provide status information directly to the sample collection organization. Sampling device status, geo-localization, user's name may as well be communicated by the user directly, by his smartphone using a dedication software application (an “app” connected to the Internet or the CLOUD™), or by any other appropriate means. Starting the sampling process may be made on the own initiative of the user, or may be ordered by the health authorities of a given region.

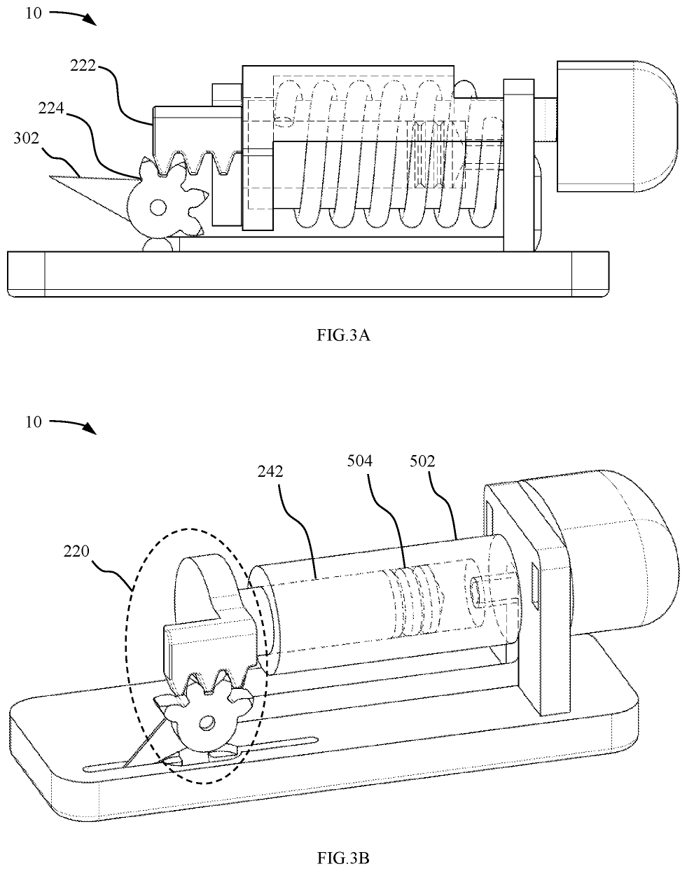

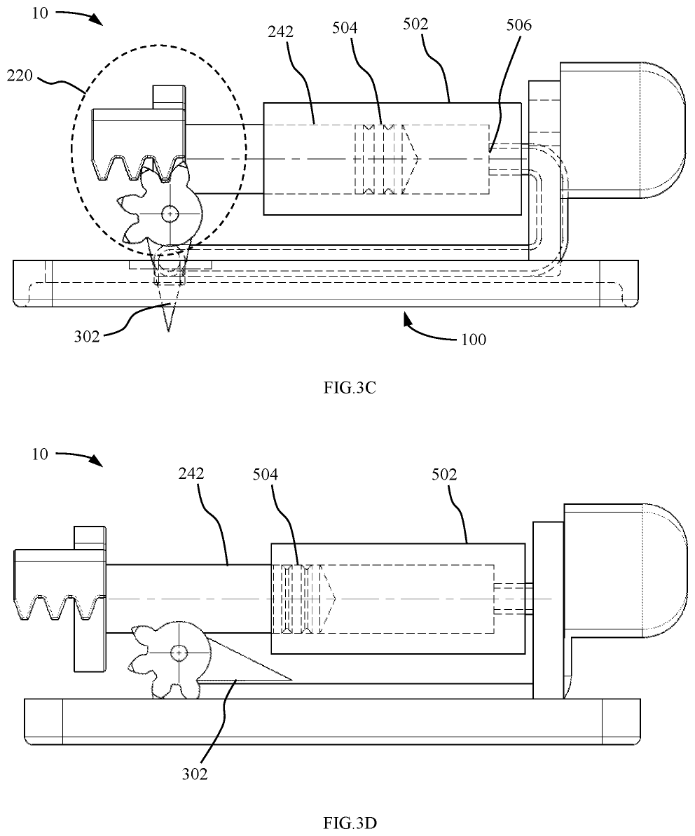

Referring now to FIGS. 3A-3D, the internal mechanism 10 of the body fluid sampling device is represented with less elements visible, to show in a clearer manner the reservoir 502, the piston 242, the plunger 504, the mechanism elements 220, 222, 224, the cutting blade 302.

Referring now to FIG. 4, the contact surface 100 of the body fluid sampling device 10 has an appropriate shape 110 so as to correspond to the shape of the area of the user's body where the sample has to be taken. The contact surface 100 may be made of soft material or include a flexible seal 120 so as to tightly fit, so that a sufficient level of vacuum is established when retracting the plunger 504 via the piston 242 to collect the body fluid sample. Optionally the contact surface 100 is protected by a film that the user removes before using the sampler, the protective film may be re-usable to close the surface 100 after sampling.

Referring now to FIG. 5, the body fluid sampling device 10 contains a channel 404 connecting at one end 402 an opening 112 in the device's contact surface 100 and at the other end 406 to the inlet 506 of the reservoir 502.

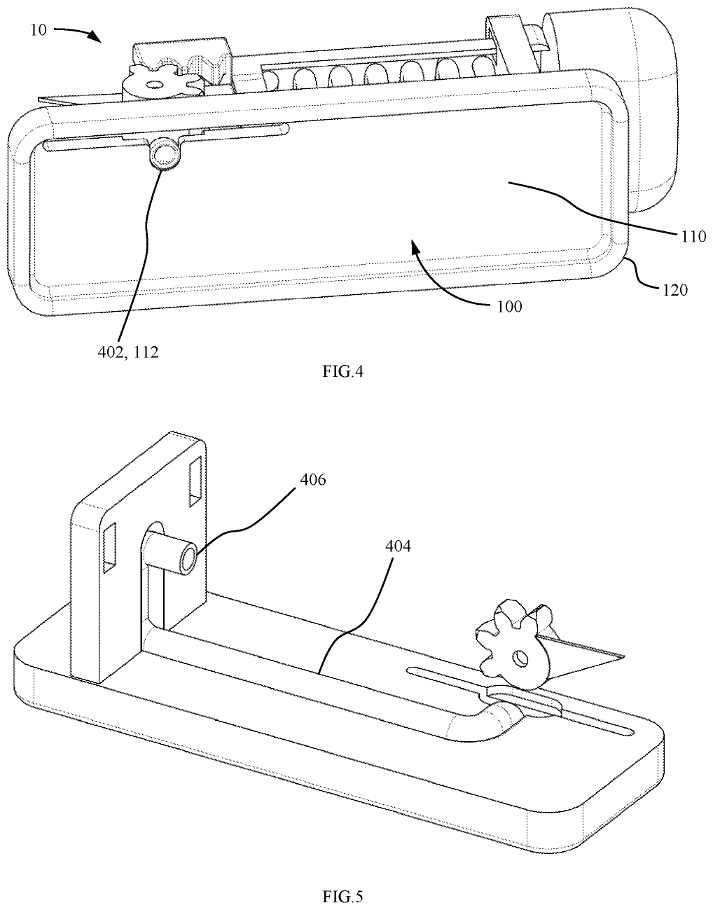

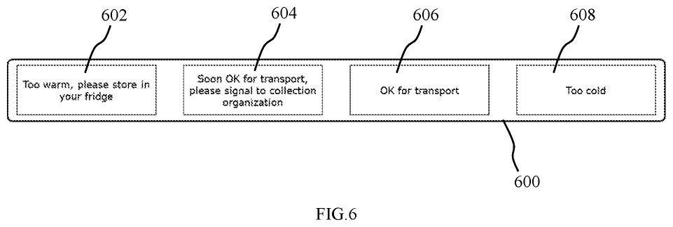

Referring now to FIG. 6, the body fluid sampling device includes a temperature monitoring display/indicator 600. Such display/indicator may consist of a printed adhesive strip 600 with several zones of different colors 602, 604, 606, 608, the color of these zones changing so as to indicate when the sampling device has reached the corresponding range of temperature, labelling of these zones may include instructions for the user. Examples of temperature ranges may be:
602: too warm (for example as long as the sampling device's temperature is above 8° C.)
604: soon ready for transport (for example when the sampling device's temperature is between 5° C. and 7° C.).
606: ready for transport (for example when the sampling device's temperature is between 1° C. and 5° C.).
608: too cold (for example when the sampling device's temperature is below a certain temperature, such as below 1° C. as another temperature range may be more appropriate for the specific fluid sample and the corresponding process and/or instructions to be given to the user.

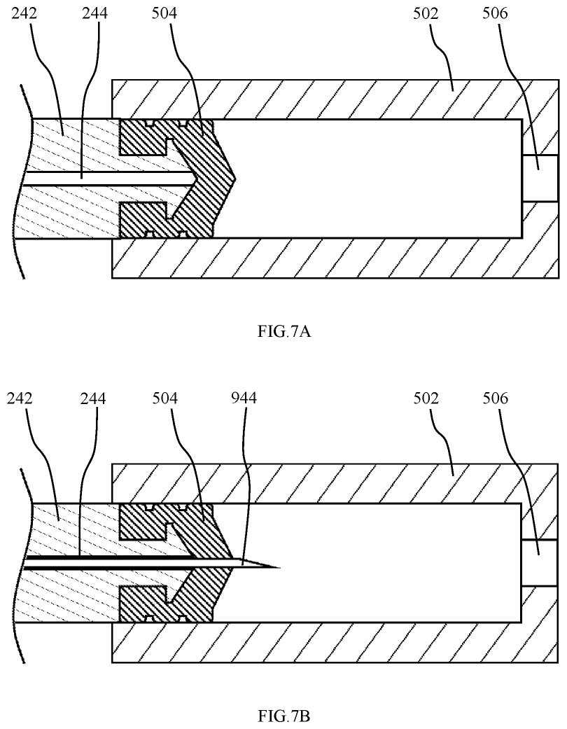

Referring now to FIGS. 7A-7B, the extraction of the body fluid sample from the sampling device is accomplished with a needle 944. An access hole 244 is provided through the piston 242, so that the needle 944 can pierce the plunger 504 and suck the body fluid sample contained in the reservoir 502.

Optionally, the body fluid sampling device advantageously includes an insulating cover or sleeve adapted to slide over the sample container so as to extend the transport time possibilities. In one embodiment, the isolating cover or sleeve is manually set in place by the user via a tab actuated by the user according to written instructions provided with the device, or automatically slid in place by a second mechanism optionally triggered by thermal contraction of an element after the device has reached a sufficiently low temperature in the refrigerator.

Any embodiment of the body fluid sampling device such as those described herein is optionally equipped with a unique identification code, and may carry an electronically readable identification tag. Depending on the circumstances, the sampling device is optionally equipped with geo-localization and long-range communication capabilities so as to be collectable without any further action from the user after the sampling process has been executed.

The body fluid sampling device according the invention is made to use standard analysis tubes as well-known in the industry, so that the tubes' content can be analyzed on standard automatized analysis equipment.

The body fluid sampling device according the invention thereby provides a non-medically trained user, such as the patients themselves, with the main functionalities of:

1. sampling a body fluid (for example blood), optionally auto-sampling (self-administered sampling);

2. optionally dispensing one or more droplet(s) of the sampled fluid for immediate analyses: and

3. providing a standard medical analysis tube filled with a sample of body fluid (for example blood) for analysis in a medical lab.

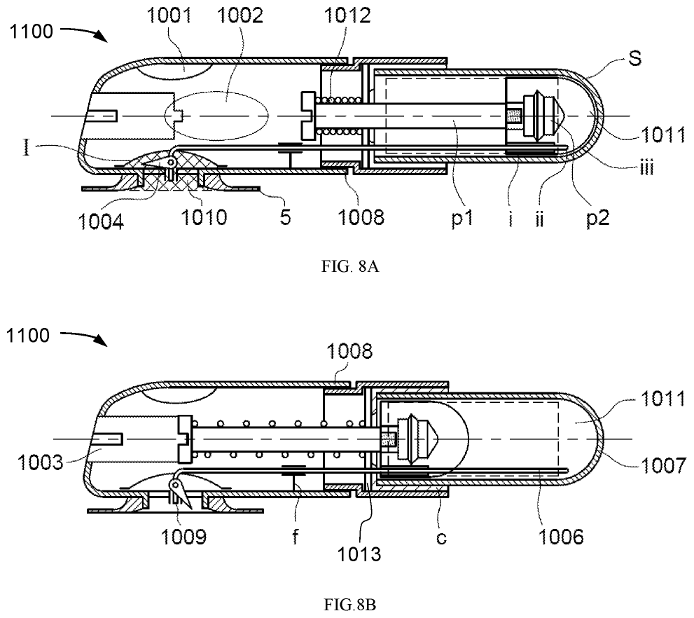

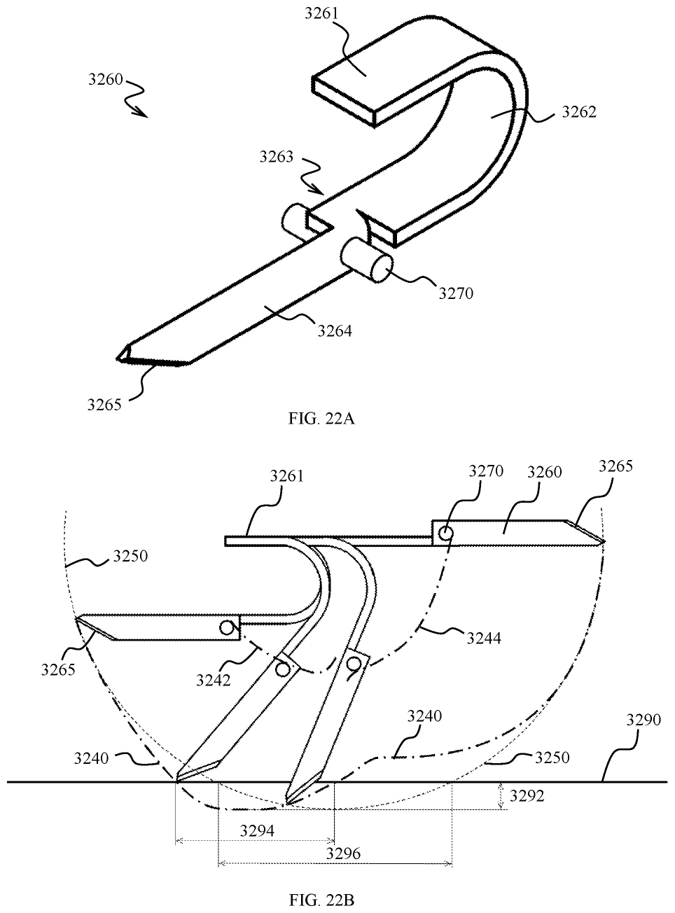

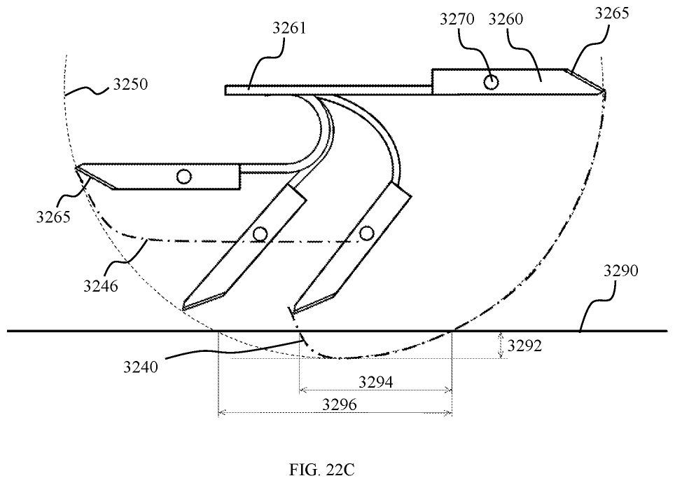

Referring now to FIG. 8A-8B, an embodiment of body fluid sampling device 1100 is presented as before use (FIG. 8A), and after the sampling (FIG. 8B). The body fluid sampling device 1100 comprises a standard medical analysis tube 1007 with its cover (c) made in standard colors and labelling (e), which preferably remains visible at all times. The device's structure preferably includes two push buttons 1002 on each side of the device 1100, whose activation, in one embodiment, must be combined with pushing the safety button 1001 in order to launch the sampling process. A combination of levers, stoppers, cams, or any other mechanical elements well known in the art triggers through a sealed sleeve I the movement of a second embodiment of one or more lancet(s) or cutting blade(s) 1004 as well as the movement of the piston p1 when the buttons 1001 and 1002 are pressed by the user. It should be mentioned that the device can have one or more cutting blades 1004 (e.g., but not limited to cutting blades 302, 5450, 5456, 3260, 3360, 3460), in principle one cutting blade for up to 500 μl, and two or more cutting blades in order to collect 1 ml or more over a duration that is acceptable to the patient. For background information, the duration of blood flow is about 30′ for 500 μl for one incision, so the same time for 1 ml with 2 incisions. The movement of the cutting blade 1004 is ensured by a dedicated spring, typically a torsion spring such as shown in FIG. 17 or element 3362 in FIG. 22A), so that it cuts an opening in the patient's skin to release capillary blood and retracts into the device's structure/housing 1008. The suction area 1010 is kept air-tight by a skirt 1005 made of flexible material such as silicone or any other appropriate material, which connects the device and the patient's skin, and a sealed sleeve I. The suction area 1010 is connected to the tube chamber 1011 via a cannula 1006, whose tip ii is similar to that of an injection needle and can pierce the plunger-septum p2 when the piston p1 is mobilized to create vacuum in the tube chamber 1011 and aspire the blood from the suction area 1010 into the tube chamber 1011. The plunger-septum p2 provides a cylindrical recess clearance i to minimize friction with the cannula during operation, a sealing lip v and a closing zone iii, that is pierced by the needle tip ii to allow the suction of the body fluid, and closes when the cannula tip ii is disengaged when the tube 1007 is removed from the device structure 1008. After the sampling, a button 1003 provides the possibility to push back the piston p1 on a short distance so as to expel a small amount of the collected blood sample back through the cannula 6 to dispense one or more droplets from the protruding end 1009 of the cannula 1006 for an immediate analysis using a home testing kit, for example. Button 1003 provides for an access to disengage the piston p1 from the plunger-septum p2, so that the plunger-septum p2 can remain engaged in the tube 1007 and keep it tightly closed when it will be released from the device's structure 8. The standard medical analysis tube 1007 is held within the devices' structure 1008 for the sampling process and the optional droplet dispensing process. The devices' structure 1008 may be made of several parts, and may contain elements that can be broken or disassembled to release the standard medical analysis tube 1007, so that it can be delivered to a lab for processing by standard medical lab analysis equipment. Before releasing the standard medical analysis tube 1007, the piston p1 must be separated from the plunger-septum p2, by, for example, turning the button 1003 to rotate the piston p1 so that it disengages from the plunger p2 via, for example, unthreading at threads x. The actuation spring 1012 is maintained within the device's structure by a washer 1013. Note that the end of the piston shaft engages with the button so as to rotate therewith when it reaches the end of the suction cycle, allowing for removal of the shaft from the plunger.

The use or operation of the device can be described as follows:
1. Typically the user holds the device between his thumb and major finger, placed on the two buttons 2, and applies his index on the safety button 1001.
2. A combination of levers, stoppers, cams, or any other mechanical elements such as well-known in the micro-mechanics art triggers the movement of the piston p1 when the buttons 1001 and 1002 are pressed by the user. At the same time, it triggers the movement of the cutting blade 1004.
3. The spring 1012 actuates the piston p1, to which the plunger-septum p2 is attached, so that the plunger impales on the needle tip ii, the tip ii piercing through the plunger, and opens the connection between the suction area 1010 and the tube chamber 1011. The cutting blade moves and cuts the skin of the patient.
4. The spring continues to pull the piston p1, so that a vacuum is created within the chamber 1011 of the medical analysis tube 1007. The patient starts bleeding, the blood is aspired through the cannula 1006 into the tube chamber 1011. The cutting blade 1004 continues its movement and retracts within the device's structure.
5. The piston p1 reaches the end of its stroke against button 1003, the suction stops, the sample is contained within the tube chamber 1011. Typically the volume of the sample is 500 μL. with one cutting blade, 1 ml with two cutting blades.
6. The user removes the device from the patient's skin, cleans the wound, applies a Band-Aid if necessary.
7. Optionally, the user uses button 1003 to expel a small part of the sample, typically 1025 μL, and uses it on an immediate test as available on the market.
8. User uses button 1003 to disconnect piston p1 from plunger-septum p2, so that plunger-septum p2 remains in the tube 1007 to keep it tightly sealed.
9. User releases the tube 1007 from the device's structure, upon removing the tube the plunger-septum p2 disengages from the cannula 1006 and closes.
10. User sends or brings the tube to a medical analyses lab to be analyzed.
11. The analysis lab analyses the blood sample and communicates the test results to the patient and/or to the relevant authorities.
12. Optionally, treatment and/or quarantine protocol is initiated to ensure that a test subject having a positive test is handled in a manner to minimize the spread of the pathogen.

In another embodiment, the body fluid sampling device according the invention may include integrated analysis functions. The sample may be collected on the skin or on a mucosa, but the sample may have to be collected through the skin/mucosa, in such case the sampler is optionally equipped with a cutting blade to open the skin/mucosa and release the body fluid to be sampled. A reactant is optionally added by the user on the surface of the skin/mucosa to facilitate the conservation of the sample or to enable specific analyses.

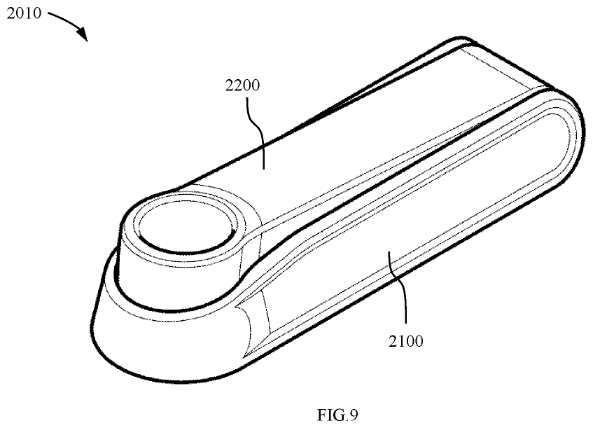

Referring now to FIG. 9, a body fluid sampling device 2010 for the collection of capillary blood from the ear lobe has a main body part 2100 and a mobile part 2200 to pinch the user-patient's ear lobe, so that the device 2010 may remain in place for the sampling process without the need of the user-patient to hold it. The body fluid sampling device 2010 contains an internal mechanism such as disclosed in U.S. 63/002,581 or U.S. 63/006,337 of the same applicant (the contents of which applications are incorporated by reference herein and relied upon) or any other appropriate mechanism which ensures a complete execution of the body fluids sampling process without any intervention other than triggering. Automatic analysis features and/or systems may be integrated in the device.

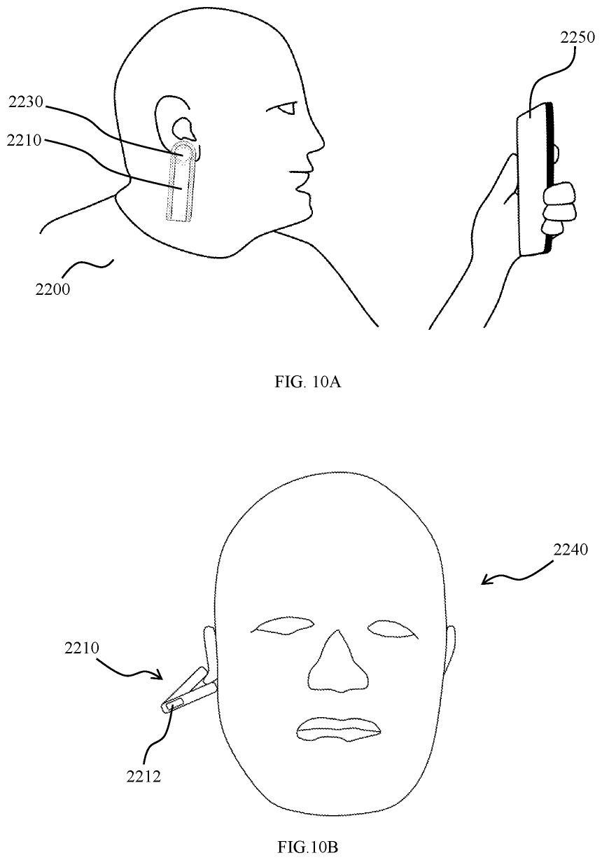

Referring now to FIGS. 10A-10B, the body fluid sampling device 2210 for the collection of capillary blood from the ear lobe 2230 of the user-patient 2200 is made with an appropriate shape, size and weight to hold by itself at the collection site 2230 and to be visible by the camera of the user-patient's smartphone 2250 together with the patient's face. An optical feature 2212 such as a QR code, a visual symbol on the device 2210 is provided to help in identifying and localizing the device and may also include a display showing the readiness, the progress and the completion of the sampling process or any other visual communication elements that the camera of the user-patent's smartphone 2250 may capture and that the application running the user-patent's smartphone 2250 may analyze and use for reinforcing the device's identification. The device 2210 includes a wireless communication system to communicate information with the user-patient's smartphone, to trigger the body fluid sampling process, and optionally to launch a sample analysis process after the sampling is completed. Information communicated between the device 2210 and the user-patient's smartphone 2250 include: device unique identification, sampling triggering, sampling process progress, sampling process completion, and/or error messages. The communication between the device 2210 and the user-patient's smartphone 2250, as well as the communication between the user-patient's smartphone and the cloud are encrypted, and may use blockchain technologies to make tampering, misuse or hacking difficult or impossible. The sampling information together with the user-patient's identification are communicated to the relevant authority for further processing.

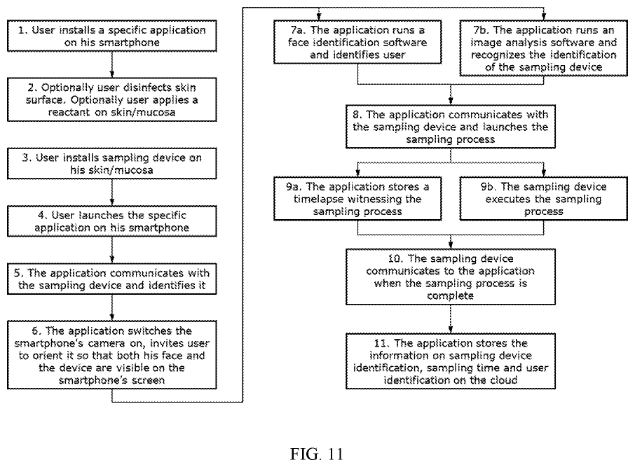

Referring now to FIG. 11, a fifth method for sampling body fluids and ensuring the identification of the user-patient includes the steps of:
1. User installing a specific application on his or her smartphone (for the first use);
2. Optionally, user disinfecting skin surface—optionally user applying a reactant on skin/mucosa;
3. User installing sampling device on his skin/mucosa;
4. User launching the specific application on his smartphone;
5. The application communicating with the sampling device and identifying it;
6. The application switching the smartphone's camera on, inviting user to orient it so that both his face and the device are visible on the smartphone's screen;
7. In parallel:
   a. The application running a face identification software and identifies user;
   b. The application running an image analysis software and recognizing the identification of the sampling device;
8. The application communicating with the sampling device and launching the sampling process:
9. In parallel:
   a. The application storing a time-lapse video as a witness to the sampling process;
   b. The sampling device executing the sampling process;
10. The sampling device communicating to the application when the sampling process is complete; and
11. The application storing information on sampling device identification, sampling time and user identification on the cloud and/or in its internal memory.

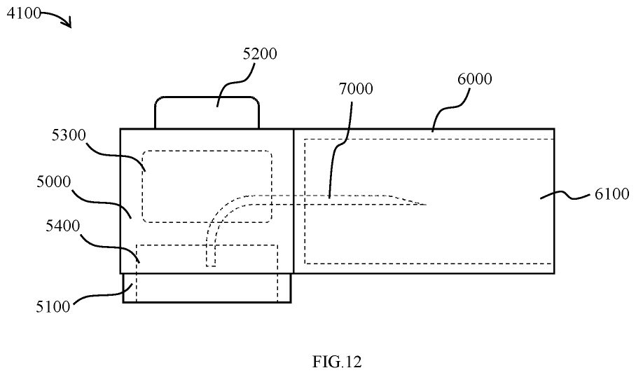

Referring now to FIG. 12, an embodiment of capillary blood sampling device 4100 according the invention has a main structure 5000 containing the suction interface 5100 adapted to be attached to the skin of the user/patient, one or more push-button(s) 5200 for the user/patient to actuate the device 4100, and a tube holder 6000 configured to receive vacuum sampling tubes such as the vacuum sampling tubes well-known in the industry for venous blood sampling. Inside of the main structure 5000 are located a suction chamber 5400, connected to the inside 6100 of the tube holder 6000 via a channel 7000, an actuation mechanism 5300 to convert the action of the user/patient pushing on the one or more buttons 5200 into actuation of the device 4100, and optional electronic and/or connectivity features such as GPS or geo-localization system, identification tag, wireless communication system, or countdown with audible feedback, etc.

The device's structure 5000 preferably includes two or more push buttons 5200 and the mechanism 5300 is configured to ensure that only the activation of all push-buttons 5200 launches the sampling process so that the risk of inadvertent launching is minimized. The mechanism 5300 may include a combination of levers, stoppers, cams, or any other mechanical elements well known in the micro-mechanics art, or may preferably be made of flexible elements that can release the sampling just by being deformed when the user/patient presses the push-button(s) 5200. The device's structure 5000 optionally includes means to expel a small quantity of the blood from the device 4100 for quick on-site analysis. A second embodiment of the cutting blade 5450 is shown in these figures, but of course, any version could be used.

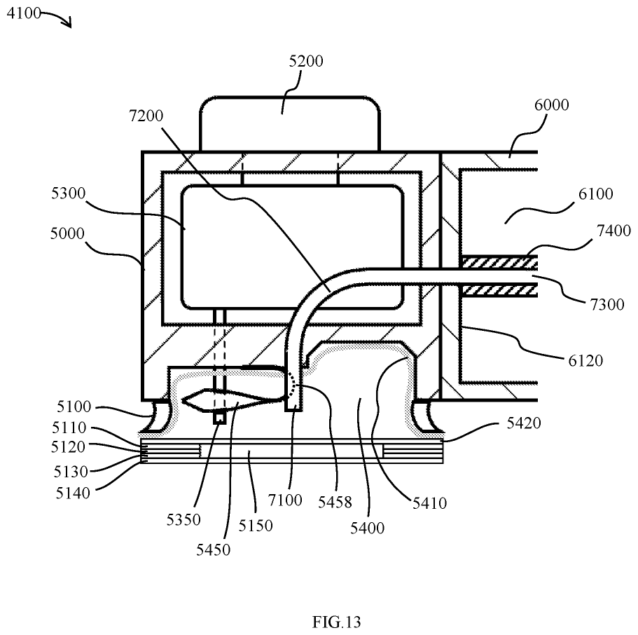

Referring now to FIG. 13, an embodiment of capillary blood sampling device 4100 according the invention is in contact with the user/patient through its suction interface 5100, the suction interface 5100 being attached to a bandage 5120 with a suitable permanent adhesive glue 5110, the bandage 5120 having on its surface facing the user/patient's skin a temporary adhesive glue 5130 adapted to hold the device 4100 unto the user/patient's skin at least for the duration of the sampling process. The temporary adhesive glue 5130 is protected before the device's use by a removable protective film 5140. The glues 5110, 5130 and bandage 5120 expose a passage 5150 to allow for blood sampling, and are air-tight on their periphery in order to ensure the build-up of vacuum between the user/patient's skin and the suction chamber 5400 for the sampling process. The bandage 5120 may have a relatively large surface compared to the surface of the suction interface, in order to ensure sufficient air-tightness with the user/patient's skin.

The suction chamber 5400 contains 1 or more cutting blade(s) 5450 of a second embodiment, in principle 1 for up to 500 μl, and 2 if 1 ml need be collected, as required depending on the sample size to be collected. For the reader's information, the duration of blood flow is about 30′ for 500 μl for one incision, and so about the same time for 1 ml using two incisions. The cutting blade 5450 has an elastic or spring portion 5458 which has been loaded at the assembly of the device 4100, and is held under tension by a mechanical finger 5350. The mechanical finger 5350 is linked to the mechanism 5300 so that the mechanism 5300 can release the cutting blade 5450 when actuated. The cutting blade is positioned so as to lacerate the user/patient's skin through the passage 5150 when released. The suction chamber is closed by an air-tight membrane 5420 made of an air-tight material that can be lacerated by the cutting blade 5450 while at the same time cutting the user/patient's skin without tearing. Optionally, the suction chamber 5400 contains an air-tight elastic lining 5410 which allows the mechanism 5300 to actuate the mechanical finger 5350 in an air-tight manner, and optionally includes means to expel a small quantity of blood for quick on-site analyses. Appropriate materials for the membrane 5420 and the optional elastic lining 5410 are well-known in the art and may include silicone, rubber, and other elastomers and/or plastics in one or more layers. The suction chamber 5400 is made so as to minimize its volume, so that the majority of the collected blood doesn't remain in the suction chamber 5400 and can be fed into the vacuum tube, and a minimal part of the vacuum provided by the tube is used to establish vacuum in the suction chamber 5400. The channel 7000 connecting the suction chamber 5400 to the inside 6100 of the tube holder 6000 may be a needle 7200 with the suction end 7100 of the needle connected to the suction chamber 5400 and the dispensing end 7300 of the needle adapted to enter the vacuum tube and bring the collected blood into the tube. Typically, the needle 7200 may be made of stainless steel, but other materials available nowadays in the industry such as other metals, composites and/or plastics may be used.

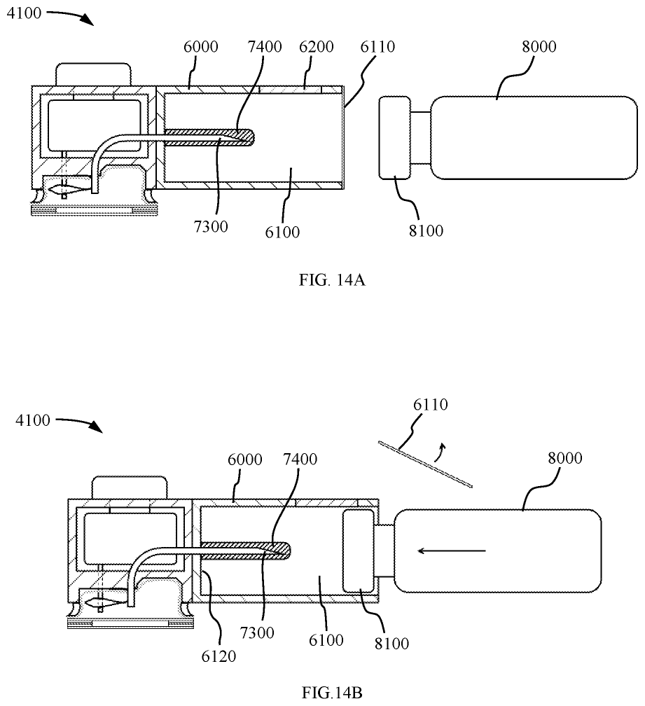

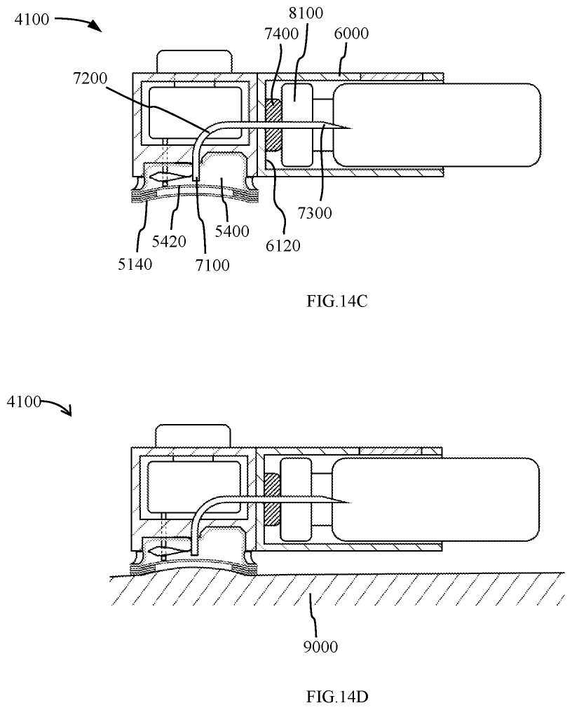

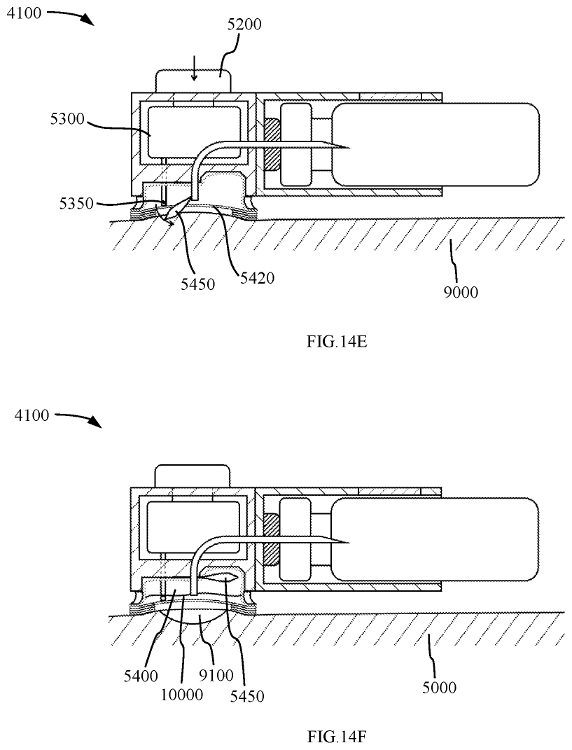

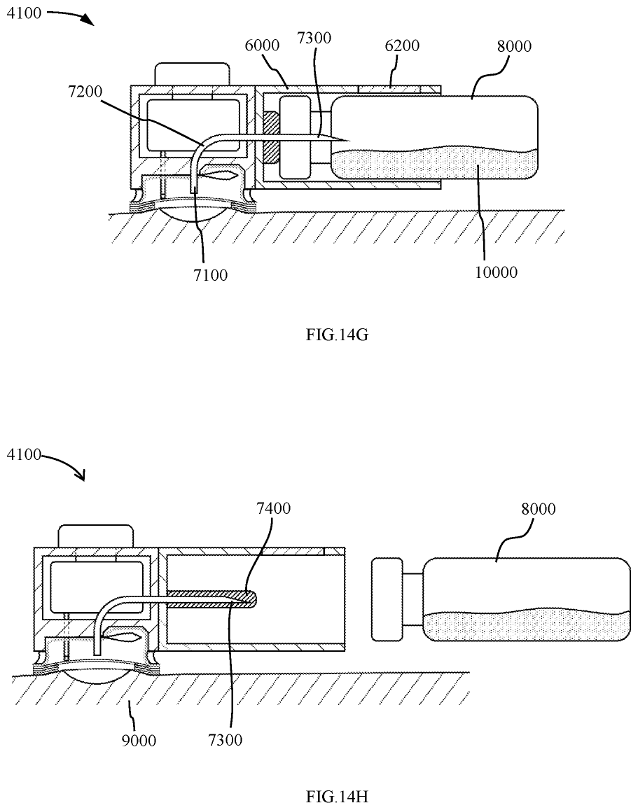

Referring now to FIGS. 14A to 14I, the functioning of the device can be described as follows:
1. See FIG. 14A: The sampling device 4100 and the vacuum tube 8000 are delivered separately to the user/patient in sterile packaging. The dispensing end 7300 of the needle is sharp so as to be able to pierce the septum 8100 (also sometimes called “rubber stopper”) of the vacuum tube 8000, and is protected by an elastic (preferably silicon or rubber) sleeve 7400. Optionally, the tube holder 6000 is closed by a removable protective film 6110. Optionally, the tube holder 6000 is made of transparent material. Alternatively, optionally, the tube holder 6000 contains a transparent window 6200 for the user/patient to see the inside 6100 of the tube holder 2000. Optionally, the rubber sleeve 7400 is made of a “self-healing” material such as well-known in the industry so that it automatically closes the dispensing end of the needle 7300 after use.
2. See FIG. 14B: The user/patient removes the optional protective film 6110, and inserts the vacuum tube 8000 in the tube holder 6000, the septum 8100 of the vacuum tube 8000 facing the dispensing end 7300 of the needle, until reaching the bottom 2120 of the inside 6100 of the tube holder 6000.
3. See FIG. 14C: When reaching the bottom 6120 of the inside 6100 of the tube holder 6000, the vacuum tube 8000 compresses the rubber sleeve 7400, stripping the dispensing end of the needle 7300, allowing the dispensing end of the needle 7300 to pierce the septum 8100 of the vacuum tube 8000 and establishing an airtight connection from the vacuum tube 8000 to the suction chamber 5400 via the needle 7200. As a result, the suction chamber 5400 is placed under vacuum.
4. See FIG. 14D: The user/patient disinfects the area of skin 9000 where the blood collection is to be made, removes the protective film 5140 and applies the device 4100 on the area where the blood collection is to be made. As a result, the device holds and seals against the user/patient's skin 9000 thanks to the bandage 5120 and the glue 5110 and 5130.
5. See FIG. 14E: The user/patient actuates the mechanism 5300 by pushing the one or more push-button(s) 5200. The mechanism then actuates the mechanical finger 5350 and releases the one or more cutting blade(s) 5450. The one or more cutting blade(s) 5450 cut(s) through the membrane 5420 and the user/patient's skin 9000, lacerating through several capillaries in the patient's skin 9000, and allowing the vacuum to access the user/patient's skin 9000.
6. See FIG. 14F: After having lacerated the membrane 5420 and the user/patient's skin 9000, the one or more cutting blade(s) 5450 terminate(s) its/their movement in a recessed area of the suction chamber 5400 out of the wound area 9100, its/their sharp edges out of the reach of the user/patient. As the wound 9100 starts bleeding, the suction chamber 5400 is progressively filled with the user/patient's blood 10000.
7. See FIG. 14G: The blood 10000 fills the vacuum tube 8000 through the suction end 7100 of the needle 7200, through the needle 7200, through the dispensing end of the needle 7300.
8. See FIG. 14H: When the vacuum tube 8000 is sufficiently filled with blood 10000, the user/patient removes the vacuum tube 8000 from the device. Indication that the vacuum tube is sufficiently filled may be provided through an electronic or mechanical timer integrated in the mechanism 5300, through a graduation on the vacuum tube which the user/patient can see through the transparent tube holder 6000, through a magnetic or capacitance-sensitive strip that is in contact with the blood, or through the transparent window 6200, or by the observation of the stopping of the blood flow when the vacuum has been exhausted, or by any other appropriate means. As the vacuum tube is removed from the tube holder 6000, the elastic sleeve 7400 is free to extend and cover the dispensing end of the needle 7300, closing the path for the blood 10000. Optionally, the user/patient inserts a further vacuum tube to collect a further blood sample, repeating steps 7 and 8.
9. See FIG. 14I: The user/patient removes the device 4100 and applies a typically separately purchased, small-wounds bandage 9200 on the wound 9100. The blood remaining in the suction chamber 5400 is retained in the suction chamber 5400 by the membrane 5420. In case of the need for a quick on-site analysis, a few drops of the blood contained in the suction chamber 5400 can be obtained by pressing on the membrane 5420. Optionally, a second mechanism (not represented) integrated in the structure 5000 provides the means to expel a small quantity of blood by squeezing the elastic lining 5410 upon pressing a push-button.
10. User sends or brings the tube 8000 to a medical analysis lab or to a point of care to be analyzed. The user/patient's own refrigerator and, optionally, a container with high thermal inertia such as that described in US application no. U.S. 63/002,581, or U.S. 63/006,337, the contents of which are incorporated herein by reference and relied upon, or a container with high thermal inertia and/or thermal insulation optionally equipped with temperature monitoring and/or signaling, may also be used to facilitate the delivery of a suitable sample to a laboratory.
11. The analysis lab analyzes the blood sample and communicates the test results to the patient and/or to the relevant authorities.
12. Optionally, treatment and/or quarantine protocol is initiated to ensure that a test subject having a positive test is handled in a manner to minimize the spread of the pathogen.

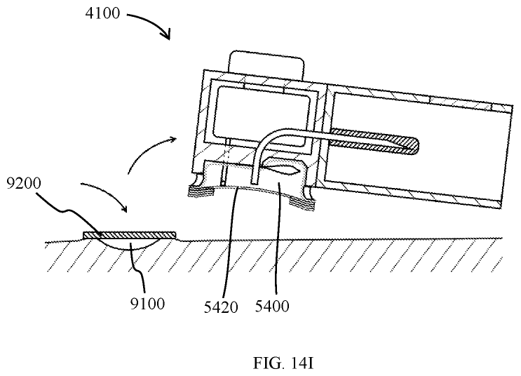

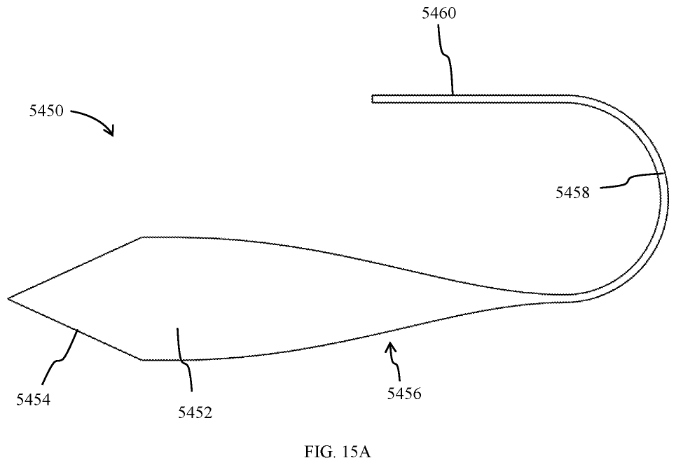

Referring now to FIGS. 15A-15C, a second embodiment of the cutting blade(s) 5450 is made of a single part, typically out of stamped sheet metal or spring steel, but may be also made of other materials, including composite materials presenting the appropriate mechanical properties. The cutting blade has an end 5460 facing the sampling device structure 5000 so that it can be attached to it, followed by an elastic zone 5458 which is bent elastically when the cutting blade is ready to be used. In this way, the cutting blade 5450 contains all the energy necessary for its movement. The elastic zone 5458 has an appropriate, preferably flat cross-section so as to provide a preferred release trajectory perpendicular to its attachment end 5460.

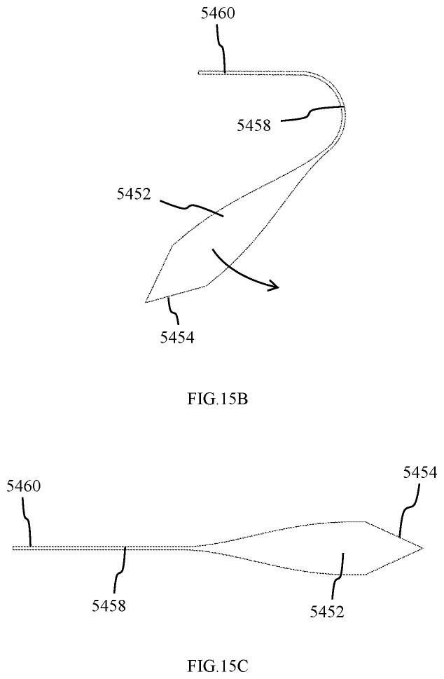

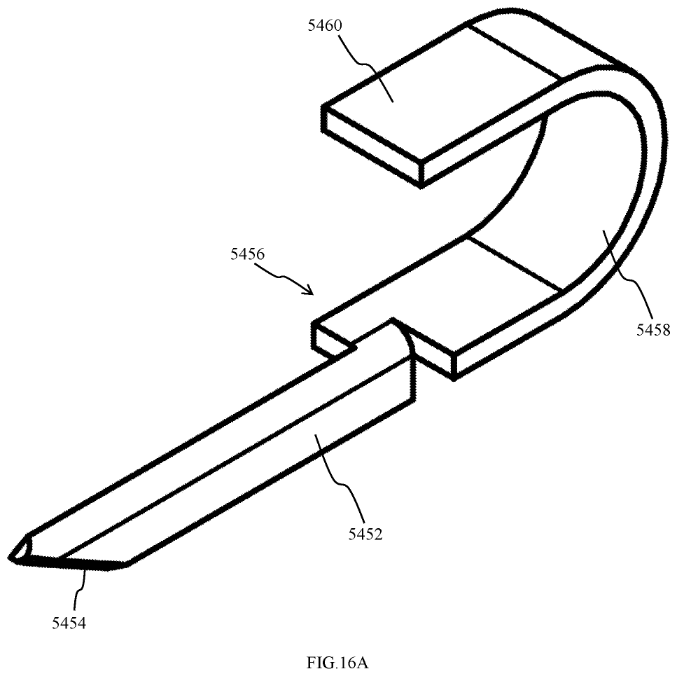

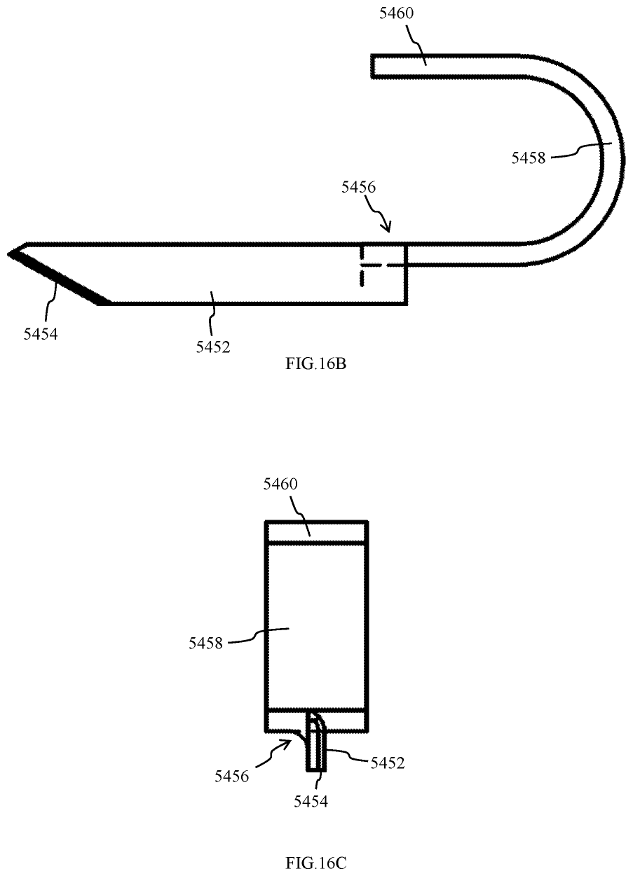

Referring now to FIGS. 16A to 16F, a third embodiment of the cutting blade 5456, formed so as to be made in a different and advantageous manner, is shown. In the above two embodiments of the cutting blade 302, 5456, the release trajectory is illustrated by a bent arrow in FIG. 15B and FIG. 16E. In one embodiment, after the elastic zone 5458, the cutting blade is twisted by 90° in the area 5456 so as to provide a blade 5452 which is in the same plane as the release trajectory.

Referring now to FIG. 17, in another embodiment, the elastic zone may take the form of a multiturn torsion spring, such as can be found in pegs. In such case, the cross-section of the blade is round and the varying stiffnesses necessary for the function are obtained by variation of the thermal treatments of the different zones of the blade. The blade 5452 has a high rigidity in the direction of the release trajectory. Towards the end of the blade 5452, a cutting edge 5454 is provided so as to lacerate the membrane 5420 and the user/patient's skin 9000. After completion of the movement of the cutting blade 5450, the cutting edge 5454 faces the same side as the attachment end 5460, away from the user/patient's reach.

In another embodiment, the device 4100 may be remotely triggered via a smartphone, optionally, following a face recognition or recognition of a QR code visible on the device. Given the device is glued on the arm, the patient has a hand free to control such remote triggering.

In still another embodiment, the capillary blood sampling system according the invention includes a patient biometric based authentication system, a device authentication system and a disposable capillary blood sampling device providing a non-medically trained user with the ability to (a) sample a capillary blood, optionally auto-sampling; (b) optionally, using one or more droplet(s) of the sampled capillary blood, to immediately analyze blood; and (c) to provide a standard medical analysis tube filled with a sample of capillary blood for analysis in a point of care or in a medical lab, the device including an interface for a vacuum tube, the vacuum tube providing suction necessary to fill the vacuum tube with the blood.

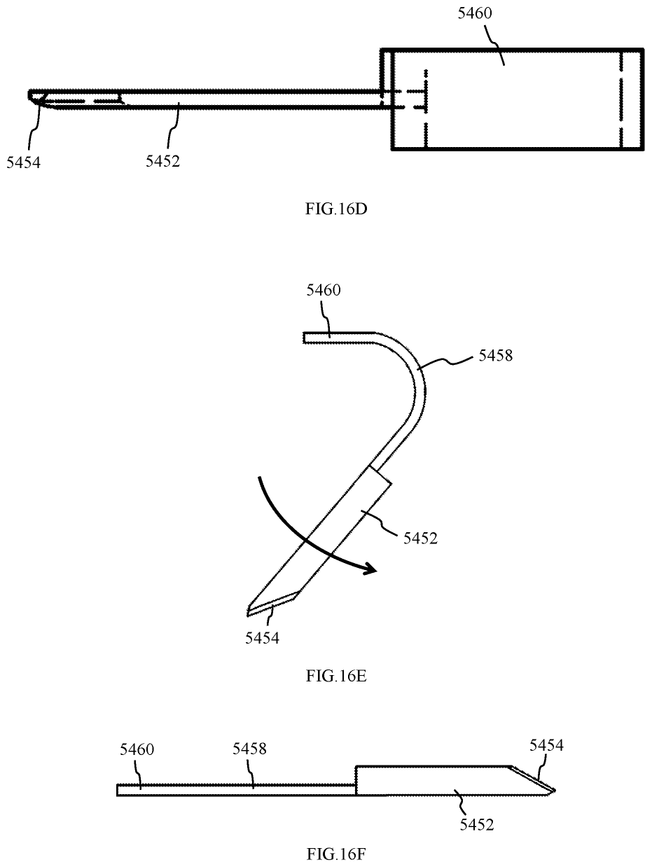

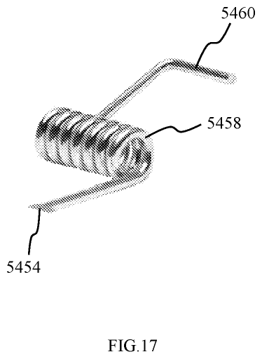

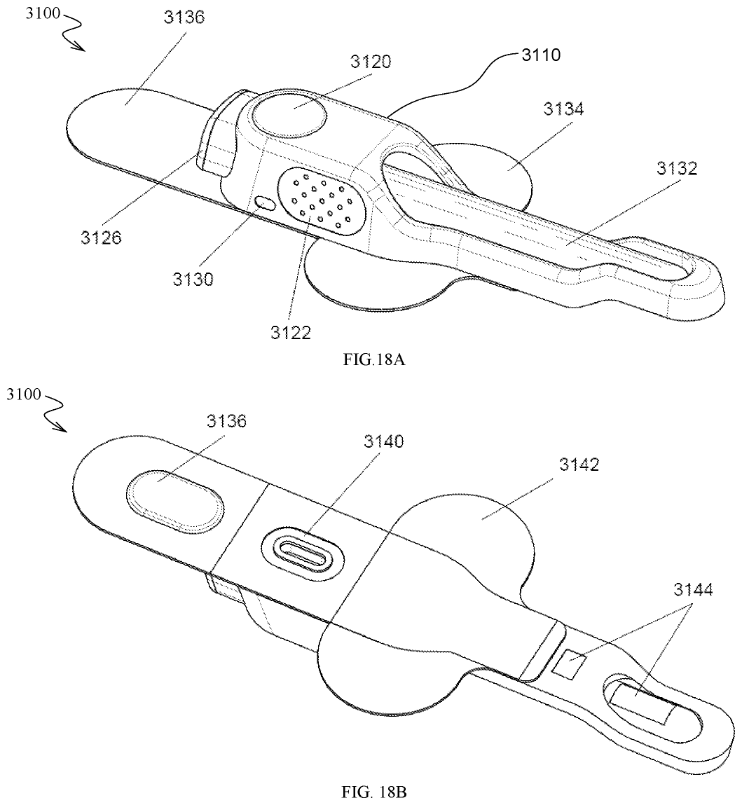

Referring now to FIG. 18A and FIG. 18B, the main features of a blood sampling device of the invention are shown. The blood sampling device comprises on its top side a grip 3122, a draw blood button 3120, a collect blood button 3126, a flash window 3130 indicating readiness for collecting blood, a blood collection tube 3132, and wing tabs 3134 for device removal as well as dressing 3136 with backer thereon. The blood sampling device 3100 further comprises blood draw reservoir 3140 with wound seal, removal wing tab 3142 with adhesive backing on and machine-readable codes 3144.

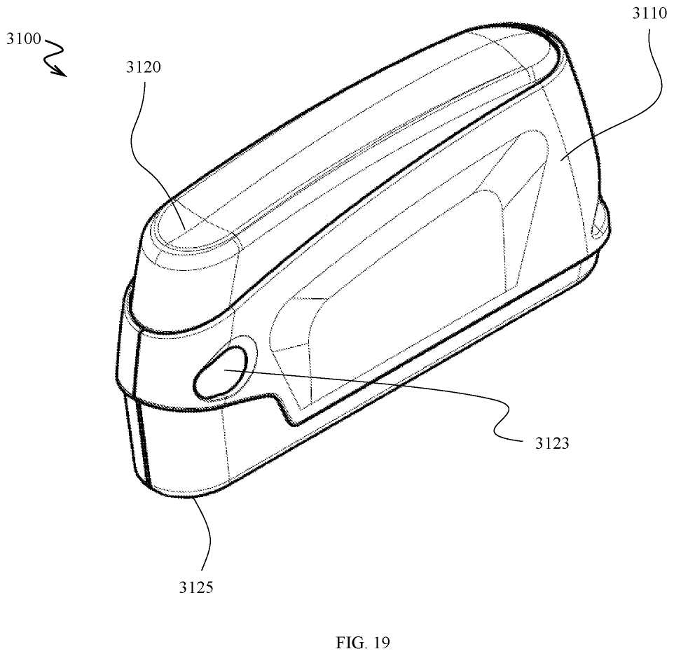

Referring now to FIG. 19, the body 3110 of the injecting device 3100 contains a mechanism (not shown) that actuates the needle of the invention in the area of the injection location 3125 when the user/patient depresses the “inject” button 3120. Such mechanism is well known in the micromechanics arts, and can contain levers, clips, springs, flexible elements, sliders, gears, cams, etc. The injecting device 3100 may further comprise a safety button 3123.

The injection device can be any suitable injection device such as those depicted in US patent applications Nos. 63/114,162, 62/864,572, 62/511,361, Ser. Nos. 15/524,748, 31/040,459, 14/235,107, and 16/930,383, the contents of which are incorporated by reference and relied upon as important disclosure in the present application.

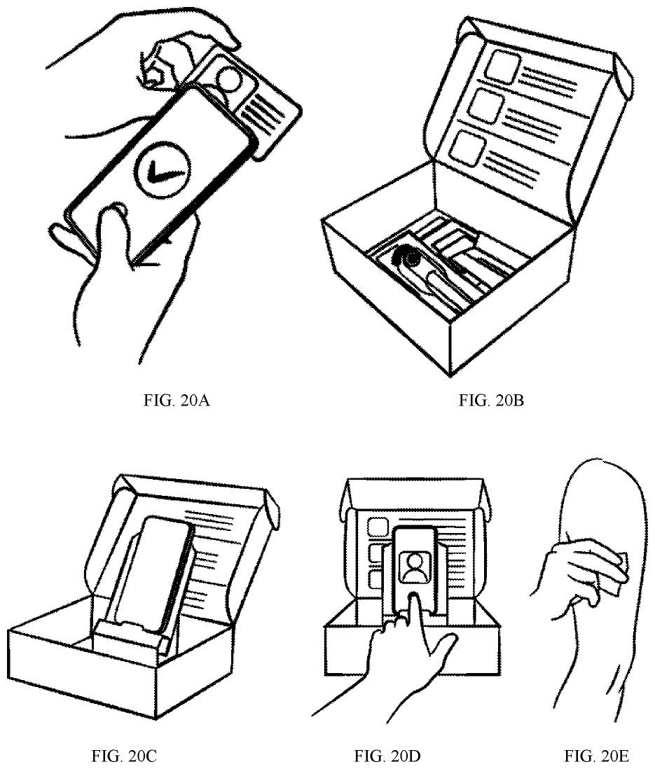

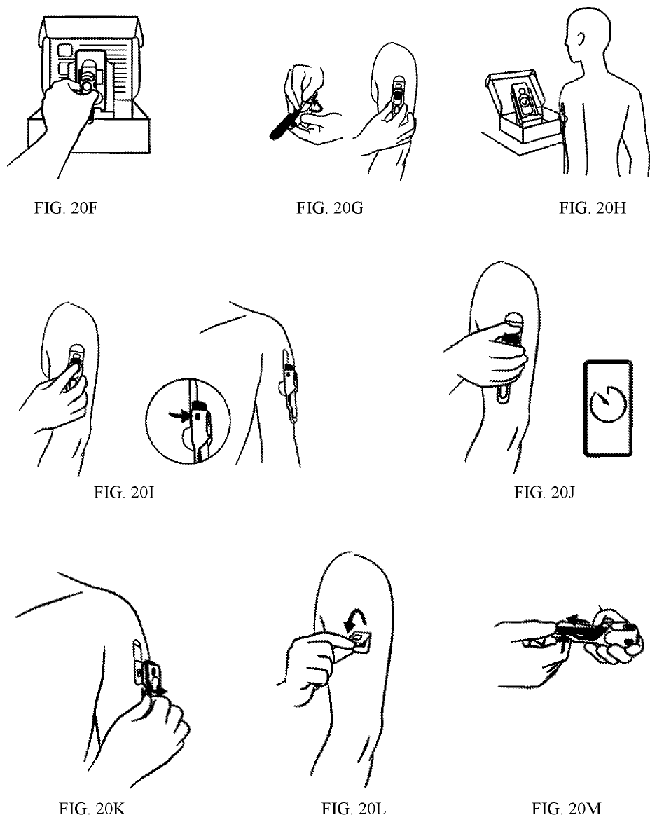

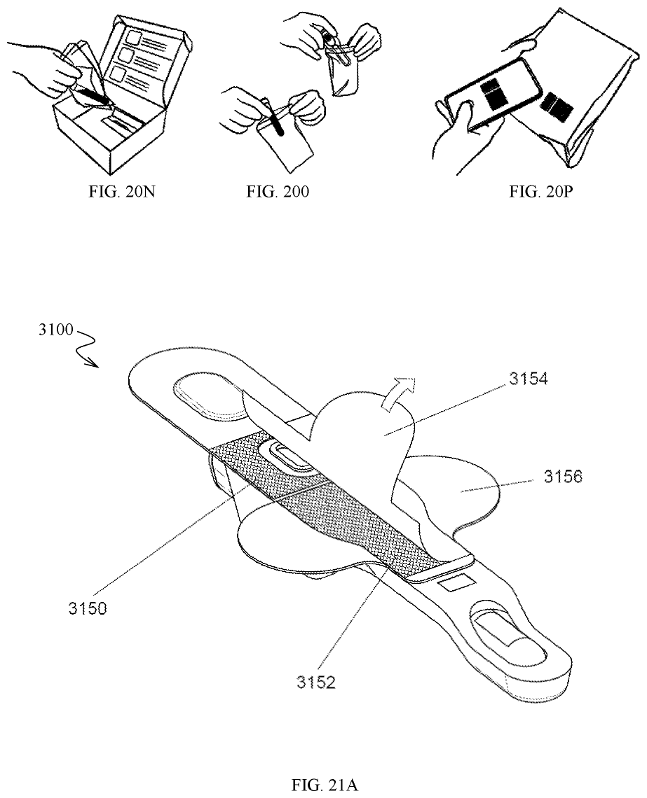

Referring now to FIGS. 20A to 20P, a sixth embodiment of the method of using the system of the invention is shown, and the specific features of the user authentication system 1900, of the device authentication system 1902, of the capillary blood sampling device 3100, 4100 and of the adhesive integrated dressing 3174 included in the capillary blood sampling device 3100, 4100 can be deducted.

FIG. 20A shows step 1 of the sixth embodiment of the method of using the system of the invention. Step 1 comprises: a) loading a dedicated App for use with the invention via scan website or prescription; b) executing a consent form; c) entering demographics information; d) photographing government issued ID or another identification means; and e) taking a selfie for association with the patient and the device as well as the sample taken.

FIG. 20B shows step 2 of the sixth embodiment of the method of using the system of the invention. Step 2 comprises: a) opening package; b) reading instructions; c) removing contents from package; d) scanning QR code on kit box to launch App and guide; e) checking contents; and f) cleaning phone with sanitary wipe included in kit.

FIG. 20C shows step 3 of the sixth embodiment of the method of using the system of the invention. The step 3 comprises: a) deploying integrated phone stand in box; b) wiping down phone with included sanitary towelette; and c) washing hands.

FIG. 20D shows the step 4 of the sixth embodiment of the method of using the system of the invention. Step 4 comprises: a) taking a selfie with phone on stand; b) starting video of self with face and arm in the frame.

FIG. 20E shows the step 5 of the sixth embodiment of the method of using the system of the invention. Step 5 comprises: a) following App guide (reading text/reviewing figures); b) preparing arm with a warm towel; c) opening alcohol towelette from kit; and d) wiping site on upper arm with alcohol, then letting dry.

FIG. 20F shows step 6 of the sixth embodiment of the method of using the system of the invention. step 6 comprises: a) removing device from kit packaging; and b) presenting QR code on device and tube to camera on stand.

FIG. 20G shows step 7 of the sixth embodiment of the method of using the system of the invention. Step 7 comprises: a) removing adhesive backing; and b) adhering device to upper arm.

FIG. 2011 shows step 8 of the sixth embodiment of the method of using the system of the invention. Step 8 comprises: confirming video of self with face device are in the frame.

FIG. 201 shows the step 9 of the sixth embodiment of the method of using the system of the invention. Step 9 comprises: a) pushing a first button; b) waiting until in flash window an indication is indicated; c) confirm the indication; and d) if no indication is indicated in prescribed time, follow guide to return kit.

FIG. 20J shows the step 10 of the sixth embodiment of the method of using the system of the invention. Step 10 comprises: a) pushing a second button; and b) waiting for timer on App to indicate tube is full with a means of communication progression.

FIG. 20K shows the step 11 of the sixth embodiment of the method of using the system of the invention. Step 11 comprises: a) pulling on wing tabs to peel the device off the arm; b) leaving dressing on arm; and c) returning device to kit box.

FIG. 20L shows the step 12 of the sixth embodiment of the method of using the system of the invention. Step 12 comprises: a) removing dressing backer revealing gauze pad and adhesive; and b) folding gauze dressing down over wound.

FIG. 20M shows the step 13 of the sixth embodiment of the method of using the system of the invention. Step 13 comprises: a) popping out end of vial from device; and b) pulling vial out of device.

FIG. 20N shows the step 14 of the sixth embodiment of the method of using the system of the invention. Step 14 comprises: a) securing sample vial in biohazard pouch from kit; and b) securing device in separate biohazard pouch from kit.

FIG. 20O shows the step 15 of the sixth embodiment of the method of using the system of the invention. Step 15 comprises: a) stopping video; b) removing phone from kit stand; c) collapsing phone stand; and d) securing both biohazard pouches in kit box.

FIG. 20P shows the step 16 of the sixth embodiment of the method of using the system of the invention. Step 16 comprises: a) sealing kit box in return shipping pouch; b) scanning QR code on shipment package; c) mailing package; d) confirming shipment; e) App notifies patient with test results.

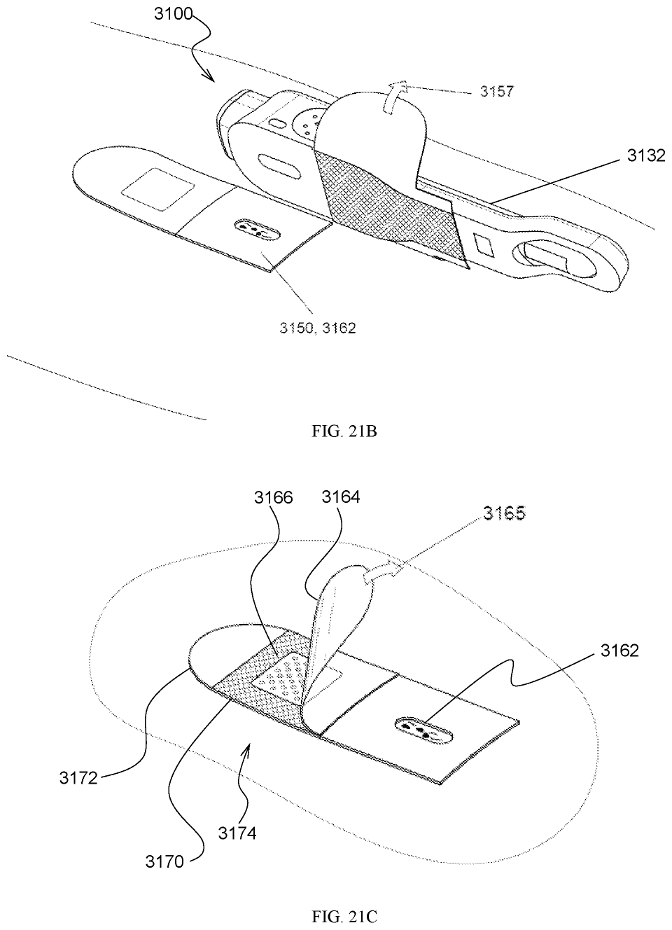

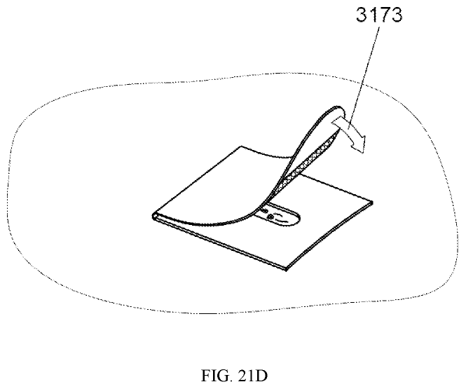

Referring now to FIGS. 21A to 21D, more details about the specific features of the adhesive integrated dressing 3174 included in the capillary blood sampling device 3100 of the invention are shown. The blood sampling device 3100 comprises an adhesive 3150 around wound site separate from the adhesive 3152 on the bottom of the device. The blood sampling device further comprises an adhesive backing 3154 which is removably located on the adhesive 3150, 3152, the adhesive backing 3154 is removed prior to the device being applied to a patient's body. Once the device is placed on a patient's arm, the device may be pulled up 3157 on a wing tab 3156 to peel the device off of the patient's skin after tube 3132 is filled. The adhesive 3150 surrounding the wound site 3162 remains behind on skin with an encompassing features to restrain the blood from flowing out of the exposed site area temporarily. Then, a dressing backer 3164 is peeled off 3165 so as to reveal gauze pad 3166 surrounded by adhesive 3170. The dressing 3174 comprise a dressing tab 3172 without adhesive. The dressing tab 3172 is pullable 3173 to fold the gauze pad onto the wound site and may be affixed to cover the wound.

As a result, the adhesive integrated dressing 3174 is adapted to ensure the attachment of the blood sampling device 3100 to the patient's skin, the air-tightness between the patient's skin and the blood sampling device 3100 during the sampling process, and the dressing of the wound after the sampling process.

In another embodiment, the vaccine and/or drug injecting device according the invention provides a non-medically trained user with the ability to (a) inject vaccines and/or drugs, optionally auto-injecting; (b) a data processing and validating system which is configured to receive data related to the vaccine and/or drug entered by the user/patient through the app, or by the HMO who received it previously (whereby the entry of the data may be performed e.g. manually or by scanning a QR-code); and (c) a system configured for sending to and, optionally, receiving from a network (such as the Internet) data.

Self-administration may be accomplished with the help of devices such as those described in PCT/US2012/048044, PCT/IB2018/000559; PCT/IB2013/000659; and PCT/IB2020/055874, the contents of which is incorporated herein by reference thereto and relied upon.

As emphasized earlier, the present invention provides means for the injection and administration of vaccines and/or drug by a non-medically trained user, the means being implemented in a vaccine and/or drug injection device. The vaccine and/or drug injection device of the invention may use one or more needle(s) as means to inject the vaccine and/or drug into the user/patient's body. For the purpose of the present disclosure, the functioning of the vaccine and/or drug injection device of the invention is described as if containing one needle, but it must be understood that the injecting device of the invention may contain more than one needles in order to increase the amount of vaccine and/or drug injected and/or to decrease the injection time. The vaccine and/or drug injecting device according the invention provides a non-medically trained user with the ability to (a) inject vaccines and/or drugs, optionally self-injecting; (b) a data processing and validating system which is configured to receive data related to the vaccine and/or drug entered by the user/patient through the app, or by the HMO who received it previously (whereat the entry of the data may be performed e.g. manually or by scanning a QR-code); and (c) a system configured for sending to and, optionally, receiving from a network (such as the Internet) data.

The device according to the present invention allows authentication for vaccines and/or drug administration. Therefore, the device according to the present invention comprises means to allow the verification of one or more of the following items:

1. The data of the physician or healthcare responsible (MED) for the vaccines/drug administration and his/her (MED) specific approval for the use of the vaccines and/or drug administration to the user/patient. The device according to the present invention is adapted to receive data related to the vaccine and/or drug entered by the user/patient through the app, or by the HMO who received it previously (whereby the entry of the data may be performed e.g. manually or by scanning a QR-code).

2. The temperature of the vaccines and/or drug administration from shipment to injection (if required). The device according to the present invention is adapted to handle information from a temperature tracer or reagent paper (typically placed in a shipment box during shipment), optionally GPS tracer (also typically placed in a shipment box during shipment). Optionally, the tracers can be returned to the vaccine and/or drug producer. Related to the device, a timer must be switched on in the app when the user/patient takes the device out of its thermal protection. Warnings have to be given by the system (app and/or device) to the user/patient related with this event.

3. Patient safety in case of patient reaction a) immediately at the time of injection (e.g. fainting); and b) after the vaccine (e.g. 15 minutes after the injection). For the aforementioned situation a), the device according to the present invention is adapted to receive from the user/patient an input (e.g. by pushing a physical button or a button in the app), so as to validate that the injection has been given. If there is no validation after a certain time after the patient has initiated the injection, an alarm is triggered to alert the MED, and, optionally, trigger an emergency procedure. For the aforementioned situation b), the system of the present invention is configured to allow the user/patient to confirm (e.g. by pushing a physical button or a button in the app) to the MED that he or she is not in need of emergency aid.

The main features of a vaccine and/or drug injecting device of the above embodiment of the invention are that the body of the injecting device contains a mechanism that actuates the needle of the invention when the user/patient depresses the “inject” button. Such mechanism is well known in the micromechanics arts, and can contain levers, clips, springs, flexible elements, sliders, gears, cams, etc.

The injection device can be any suitable injection device such as those depicted in US patent applications Nos. 63/114,162, 62/864,572, 62/511,361, Ser. Nos. 15/524,748, 31/040,459, 14/235,107, and 16/930,383, the content of which is incorporated by reference and relied upon as critical disclosure in the present application.

Capillary blood sampling devices for non-medically trained users of the current art usually create the wound in the patient's skin by perforation with one or more needles, or even without perforation, which only allows for the collection of relatively small volumes of fluid, typically less than 150 μl in 5-10 minutes.

In another embodiment, the present invention provides means for the capillary blood sampling device 10, 1100, 2010, 2210, 3100, 4100 to make a significantly larger cut than what is usually known in existing capillary blood sampling devices, so that a significantly larger amount of blood, typically more than 500 μl, preferably 1 ml can be collected over a reasonable period of time, typically less than 15 minutes, preferably less than 10 minutes. The capillary blood sampling device of the invention uses one or more cutting blade(s) 1004 (e.g., but not limited to cutting blades 302, 5450, 5456, 3260, 3360, 3460), instead of one or more needle(s), so that the user/patient's skin is lacerated instead of punctured. For the purpose of the present disclosure, the functioning of the sampling device of the invention is described assuming it contains one cutting blade, but it must be understood that the sampling device of the invention may contain more than one cutting blade in order to increase the amount of blood collected and/or to decrease the blood collection time. Moreover, the current invention provides cutting solutions that favor a quick healing of the wound after the blood sampling is complete. The purpose of the invention is therefore to create a wound in the user/patient's skin that has an optimal depth for cutting as many capillaries as possible, while avoiding unnecessary wound width and length so that the natural healing of the wound can happen as fast as possible after the blood collection. The ideal cutting depth may vary as function of the patient's age, gender, ethnical group and/or health condition, as a result several adapted versions of sampling devices may be provided. Typically ideal cutting depth is between 1 mm and 2 mm.

Referring now to FIG. 22A to FIG. 22C, in a fourth embodiment the cutting blade 3260 of a device of the invention is made of a single part, typically out of sheet metal or spring steel, but may be also made of other materials, including composite materials presenting the appropriate mechanical properties. The cutting blade 3260 has an end 3261 facing the sampling device structure so that it can be attached to it, followed by an elastic zone 3262 which is bent elastically when the cutting blade is ready to be used. In this way, the cutting blade 3260 contains all the energy necessary for its movement, and the user/patient only needs to release it by depressing the draw blood button 3120. The elastic zone 3262 has an appropriate, preferably flat cross-section so as to provide a preferred release trajectory perpendicular to its attachment end 3261. In one embodiment, after the elastic zone 3262, the cutting blade is twisted by 90° in the area 3263 so as to provide a blade section 3264 which is in the same plane as the release trajectory. The blade section 3264 has a high rigidity in the direction of the release trajectory.

Referring now to FIG. 17, the elastic zone may take the form of a multiturn torsion spring, such as can be found in clothes pins, in such case the cross-section of the blade is round and the varying stiffnesses necessary for the function are obtained by variation of the thermal treatments of the different zones of the blade. Towards the end of the blade section 3264, a cutting edge 3265 is provided so as the lacerate the user/patient's skin 3290. After completion of the movement of the cutting, edge 3265 faces away from the user/patient's reach. Optionally the cutting blade 3260 includes a finger 3270 that interacts with the device's structure elements 3242, 3244, 3246 in order to bias the natural release trajectory 3250 of the cutting blade 3260 when released to obtain a modified trajectory 3240.

Typically, the natural release trajectory 3250 of the cutting blade 3260 is substantially circular, elliptical or spiral. As a result, the laceration in the patient's skin 3290 is substantially circular and with a relatively large radius, and the wound length 3296 is relatively long for a small portion at the desired depth 3292. When elements of the device's structure 3242, 3244, 3246 interact with the finger 3270 of the cutting blade 3260, the resulting trajectory 3240 of the cutting blade 3260 is modified so that the resulting laceration of the patient's skin 3290 has a steeper dive and retraction path, resulting in a shorter wound length 3294 for a longer proportion of the wound at the desired depth 3292. The modified trajectory 3240 allows for a larger volume of capillary blood to be collected, for a globally smaller wound, favoring a quicker healing of the wound after the blood collection.

Referring now to FIG. 22B, as an example, the element of the device's structure 3242 is made to locally extend the radius of natural release trajectory 3250, and the element of the device's structure 3244 is made to locally shrink the radius of the natural release trajectory 3250.

Referring now to FIG. 22C, a further example the element of the device's structure 3246 is made to locally shrink the radius of natural release trajectory 3250.

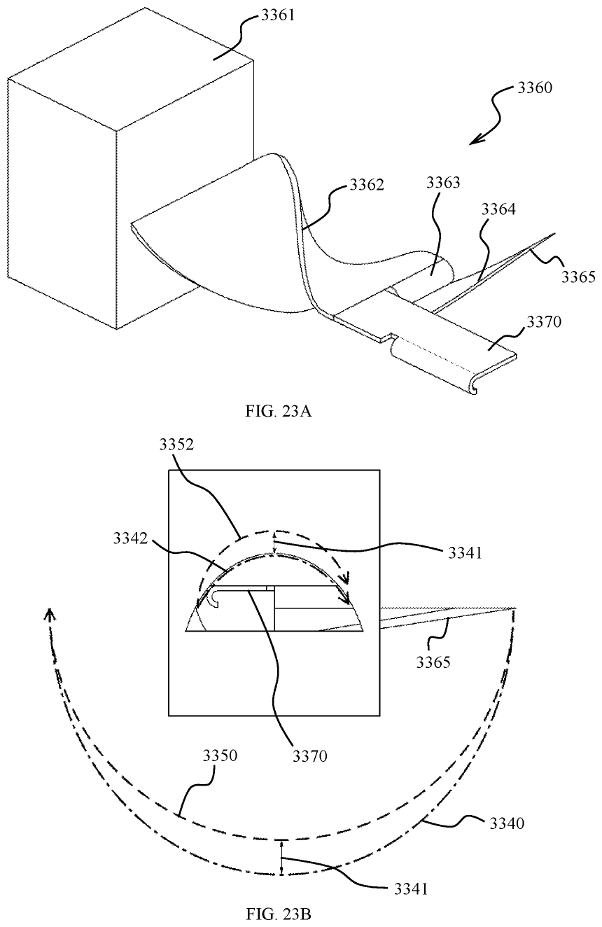

Referring now to FIG. 23A to FIG. 23B, in a fifth embodiment the cutting blade 3360 of a device of the invention is made of a single part, typically out of sheet metal, but may be also made of other materials, including composite materials presenting the appropriate mechanical properties. The cutting blade 3360 has an end 3361 facing the sampling device structure so that it can be attached to it, followed by an elastic zone 3362 which is twisted elastically when the cutting blade 3360 is ready to be used. In this way, the cutting blade 3360 contains all the energy necessary for its movement, and the user/patient only needs to release it by depressing the draw blood button 3120. The elastic zone 3362 has an appropriate, preferably flat cross-section so as to provide a preferred, planar natural release trajectory 3350. In one embodiment, after the elastic zone 3362, the cutting blade is twisted by 90° in the area 3363 so as to provide a blade section 3364 which is in the same plane as the release trajectory. The blade section 3364 has a high rigidity in the direction of the release trajectory. Towards the end of the blade section 3364, a cutting edge 3365 is provided so as to lacerate the user/patient's skin (not represented). After completion of the movement, the cutting edge 3365 faces away from the user/patient's reach. Optionally, the cutting blade 3360 includes a finger 3370 that interacts with the device's structure element 3342, in order to bias the natural release trajectory 3350 of the cutting blade 3360 when released, generating a modified release trajectory 3340.

Typically the natural release trajectory 3350 of the cutting blade 3360 is substantially circular or spiral. As a result, the laceration in the patient's skin is substantially circular and with a relatively large radius, and the wound length is relatively long for a relatively small portion at the desired depth. When an element of the device's structure 3342 interacts with the finger 3370, the resulting trajectory 3340 of the cutting blade 3360 can be modified so that the resulting laceration of the patient's skin has a steeper dive and retraction path, resulting in a shorter wound length, and a longer proportion of the wound at the desired depth. The modified trajectory 3340 allows for a larger amount of blood volume to be collected, for a generally smaller wound, favoring a quicker healing of the wound after the blood collection. As an example, the element of the device's structure 3342 is made to shrink locally the radius 3352 of the natural release trajectory 3350 by an offset 3341 on a part of the release trajectory 3340. In addition, the element of the device's structure 3342 can be made with a more complex shape so as to provide a more elaborated modified trajectory 3340.

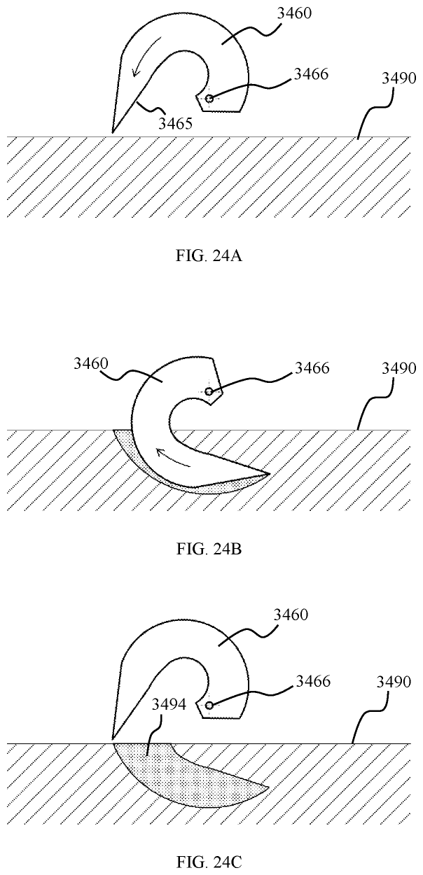

Referring now to FIG. 24A to FIG. 24C, in a sixth embodiment the cutting blade 3460 of a device of the invention is made of a single part, typically out of sheet metal, but may be also made of other materials, including composite materials presenting the appropriate mechanical properties. Referring now to FIG. 24A, the cutting blade 3460 has a rotative attachment 3466 to the device's structure and a cutting edge 3465, which is kept in retracted position prior to the cutting process.

Referring now to FIG. 24B, when actuated the cutting blade 3460 engages into the patient's skin 3490 in a rotative movement, but the rotative movement is limited by the structure so that the patient's skin is not completely lacerated.

Referring now to FIG. 24C, after the laceration the cutting blade 3460 is retracted in reverse rotative movement, leaving a wound under the patient's skin that is much smaller than if the laceration had been made completely. As a result, a larger amount of blood volume can be collected thanks to the deep laceration, but at the surface of the patient's skin 490, the opening is smaller, favoring a quicker healing of the wound after the blood collection.

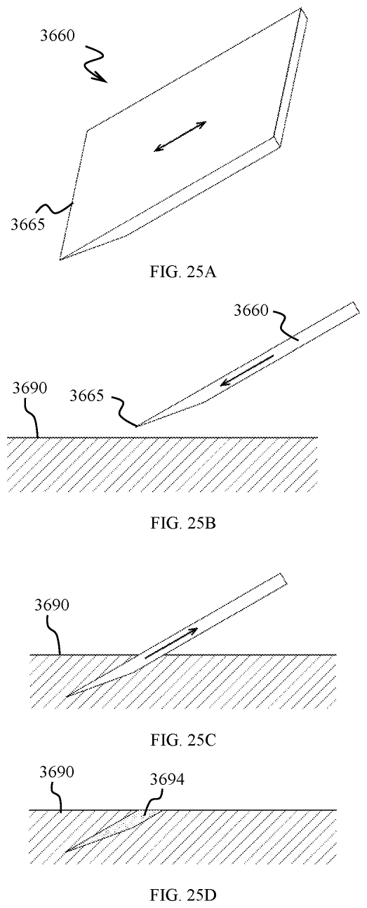

Referring now to FIG. 25A to FIG. 25D, in a fifth embodiment the cutting blade 3660 of a device of the invention may be a standard scalpel blade or any other rigid blade, typically out of sheet metal, but may be also made of other materials, including composite materials presenting the appropriate mechanical properties. The cutting blade 3660 has a cutting edge 3665, positioned substantially parallel to the skin of the user/patient 3690. The cutting blade is guided in a linear movement by the device's mechanism (not shown) in order to penetrate the user/patient's skin at a substantially non-orthogonal angle (FIG. 25B). In this way, the cutting edge 3665 penetrates completely in the skin of the user/patient, reaching a substantially uniform depth along all its length, creating a substantially rectangular wound, oriented non-orthogonally in the user/patient's skin (FIG. 25C). When the cutting blade is retracted (FIG. 25D), the flap generated by the non-orthogonal wound closes naturally the wound's entrance, favoring a quicker healing of the wound after the blood collection.

In another embodiment, the invention provides for verification of self-administered medical processes. An objective of this embodiment is to make sure the user/patient is identified:
For blood sampling: to make sure the blood in the sampling tube(s) is the blood of this patient
For injection: to make sure the injection is made in this patient.
   What may be at stake here is:
   For blood sampling
      Reliable blood sampling;
      Authorization to work, to travel, to be in contact with family in times of epidemics;
      Authorization to use a certain treatment;
      Automatized detection of epidemics by large-scale sampling programs;
      Verification of the efficiency of a given treatment (payment subject to treatment success);

For Injections: authorization to work, to travel, to be in contact with family after a vaccine/treatment has been taken may be at stake. In addition, keeping personal immunization record up to date and automatized monitoring of large-scale vaccination programs is at stake. Still further, payment of a treatment may be at stake, contingent on vaccination and only if fully administered. Therefore, making a secure patient & process verification is crucial.

In another aspect, verification of self-administered medical processes is important. For example, use of a smartphone application may be made in order to make a video (or a time-lapse) where the patient's face as well as the process itself are visible in the video all along the process duration. A comparison of the patient's ID with the patient's face may be made. The patient's face recognition and whole process may be observed. The system could be configured to launch the process only when all ID's are confirmed. Automatized handling of the logistics (sampling tube collection & transport, treatment re-supply, etc.) may be implemented. In the case that the smartphone app is configured to analyze the video in real-time, step-by-step instructions may be provided to the patient in real-time, while the process is being executed. Other features could be included such as automated monitoring of process performance. Despite the potential, there will always be challenges to deal with, particularly in degraded conditions, when dealing with insufficient network coverage at the moment of running the process. Optionally, temporary network coverage may be provided via drones/balloons for the duration of the treatment campaign. It would be best however, if the App is able to run independently of network coverage. Saving all data on the smartphone and making verifications at a later stage may also be desirable. Respecting private data regulations is an issue of course. The App may be configured to run a first analysis and then an encryption routine to make private data unreadable, with full video optionally saved for later use in case of need (e.g. formal proof in a court).

Main components of the system include a smartphone or laptop/computer or similar and camera (may be included in the smartphone/laptop/computer), the application to be run by the smartphone or laptop/computer; the device for running the injection/sampling process, which may be re-usable. In addition, the treatment to be injected/one or more empty tubes/vials to contain the collected blood sample(s) (may be under vacuum).

As for some key features of the system of the invention, the app must be able to read treatment container/sampling tube's unique ID using for example a standard bar-code/QR-code. The App must be able to identify key process steps. The device may need to include automatized wireless signal emission (information: ID/process started/process ongoing/process finished/error). Such signal may be visible (e.g. blinking/colored LEDs) for easy interpretation in the video. The device of the invention may optionally include visible features/landmarks for easier orientation verification in the video. The App should be able to launch the treatment process. The device is optionally equipped with remote triggering feature and advantageously includes a unique ID.

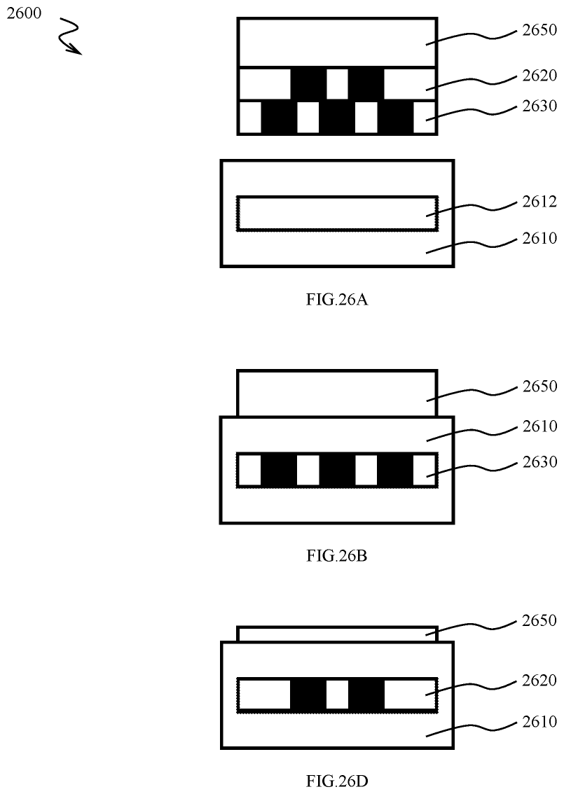

Referring now to FIG. 26A to FIG. 26C, visible features 2600 integrated in the device 2610 of the invention that can be recognized easily may optionally take the form of the combination of different high-contrast patterns 2620, 2630 applied on a mobile part (in this example a button 2650) and visible through a window 2612 only one at a time. As a result, predefined positions of the mobile part (button 2650 in high position in FIG. 26B, button 2650 in low position in FIG. 26D) can be easily recognized by the human observer, or easily identified by an image analysis software.

The invention may have other uses. For example, it may be applied to another medical treatment other than an injection (the device may be a pills distributor). It may be applied to filling a voting form at home, signing documents, proving one's ID during a teleconference, or taking a remote exam.

The invention can be summarized as including the following feature sets:

1. A disposable body fluid sampling device providing a non-medically trained user with the functionalities of (a) sampling a body fluid (for example blood), optionally auto-sampling; (b) optionally dispensing one or more droplet(s) of the sampled fluid for immediate analyses; and (c) providing a sample containment chamber filled with a sample of body fluid (for example blood) for analysis in a medical lab.
2. The disposable body fluid sampling device of feature set 1, wherein the sample containment chamber is a standard medical analysis tube.
3. A method using the body fluid sampling device of feature set 1, wherein, if the test subject tests positive for a pathogen, treatment and/or quarantine protocol is initiated to ensure that a test subject having a positive test is handled in a manner to minimize the spread of the pathogen.
4. A method for using the device of feature set 1 to collect and potentially analyze a sample of body fluids, for example blood, while ensuring the identification of the individual from whom the body fluid(s) have been sampled.
5. A disposable body fluid sampling device of one of the feature sets 1 or 2, having a sample containment chamber made of a material having a thermal inertia permitting the maintenance of sample temperature over a known period of time.
6. The disposable body fluid sampling device of feature set 5, wherein the thermal inertia is selected to provide a known period of time of storage in an ambient environment sufficient to allow non-refrigerated transport to a collection point.
7. The device of the above feature set wherein the known period of time is within a range of 1 hour to 2 hours under normal ambient conditions, and preferably within a range of 1 hour to 6 hours, and more preferably within a range of 1 hour to 8 hours.
8. A disposable body fluid sampling device of feature set 5 including a thermally insulating sleeve configured to be manually or automatically triggered to slide over the sample container chamber.
9. The disposable body fluid sampling device of the above feature set wherein the thermal inertia is selected to provide a known period of time sufficient to allow non-refrigerated transport to a collection point.
10. The device of the above feature set wherein the known period of time is within a range of 1 hour to 2 hours under normal ambient conditions, and preferably within a range of 1 hour to 6 hours, and more preferably within a range of 1 hour to 8 hours.
11. A disposable capillary blood sampling device of feature set 1, the device including an interface for and a vacuum tube, the vacuum tube providing suction necessary to fill the vacuum tube with the blood.
12. A cutting blade made for making a laceration in the skin of a user/patient for a disposable capillary blood sampling device of feature set 11, wherein the cutting blade construction is made in one piece of material and provides the energy and the guiding for its movement.
13. A device and app combination for drug or vaccine injection which provides a non-medically trained user with the ability to perform a self-injection adapted to interact with the app connected, preferably in a wireless manner, to the Internet optionally via the Cloud, the app including means to allow for verification of the patient's ID and/or the particular device used, the combination including at least the following:

a) access to data storage adapted to store data of the physician or healthcare responsible (MED) for the vaccines/drug administration and his/her specific approval for the use of the vaccines and/or drug administration to the user/patient, wherein the combination is adapted to receive data related to the vaccine and/or drug entered by the user/patient, or by the HMO who received it previously (whereby the entry of the data may be performed e.g. manually or by scanning a QR-code);

b) a recording means adapted to record temperature of the vaccines and/or drug administration from shipment to substantially the time of injection as required;

c) patient safety means adapted to be activated in case of patient reaction
i) immediately at the time of injection (e.g. fainting); and/or
ii) after the vaccine (e.g. 15 minutes after the injection), wherein, for aforementioned situation i), the combination according to the present invention is adapted to receive from the user/patient an input,

wherein further, the device is adapted to be shipped in thermal protection and the combination optionally including a temperature tracer or reagent paper (typically placed in a shipment box during shipment), and a GPS tracer (also typically placed in a shipment box during shipment) which tracks storage temperature during shipment.

14. The system of feature set 13, wherein the system is adapted to receive input by a user pushing a physical button or a button in the app, such to validate that the injection has been given.
15. The system of the above feature set, wherein, if there is no validation after a certain time after the patient has initiated the injection, the system is adapted to trigger an alarm to alert the MED, and, optionally, trigger an emergency procedure.
16. The system of feature set 13, wherein, for the aforementioned situation ii), the system of the present invention is configured to allow the user/patient to confirm (e.g. by pushing a physical button or a button in the app) to the MED that he or she is fine.
17. The system of any one of the feature sets 13-16, wherein the tracers are adapted to be returned to the vaccine and/or drug producer.
18. The system of feature set 13, wherein a timer is provided in the app which is configured to be switched on in the app when the user/patient takes the device out of its thermal protection, wherein warnings have to be given by the combination to the user/patient related with this event.
19. A device for capillary blood sampling provides a non-medically trained user with the ability to sample capillary blood, optionally analyze blood using an analysis device, and fill a sample tube with the blood, the device including:
(a) a sampling mechanism for sampling capillary blood, optionally auto-sampling;
(b) optionally, the analysis device, which using one or more droplet(s) of the sampled capillary blood, is configured to immediately analyze the blood; and
(c) a filling mechanism which is configured to fill a standard medical analysis tube with a sample of capillary blood for analysis in a point of care or medical lab,
wherein the device including a vacuum tube and an interface therefor, the vacuum tube providing suction necessary to fill the vacuum tube with the blood.
20. A device according to one of feature sets 1-11 or 19, containing one or more cutting blades for lacerating the user/patient's skin.
21. A device according to the feature set 20, wherein the one or more cutting blades are cutting blades wherein the trajectory of which is configured to be substantially non-circular as it passes through the patient's skin, providing a wound shape that optimizes the number of capillaries cut and favoring the quick healing of the wound after blood collection.
22. A device according to one of the feature sets 19-21, containing one or more cutting blades where the trajectory of the one or more cutting blades is configured to be substantially non-orthogonal in the patient's skin, providing a wound shape that optimizes the number of capillaries cut and favoring the quick healing of the wound after blood collection.
23. A device according to the feature set 22, where the trajectory of the one or more cutting blades is configured to have a limited rotation, cutting the skin essentially below its surface.
24. A device according to the feature set 22, where the trajectory of the one or more cutting blades is configured to have a substantially linear movement, substantially non-orthogonal to the user/patient's skin.
25. A method for capillary blood sampling providing a non-medically trained user with the ability to sample capillary blood, analyze blood and fill a sample tube with the blood, the method including the steps of:
(a) using the device of any one of feature sets 1, 4-11, 13-14, sampling capillary blood, optionally auto-sampling the blood;
(b) optionally, using one or more droplet(s) of the sampled capillary blood, immediately analyzing the blood and reading characteristics such as blood type; and
(c) filling a sample containment chamber with a sample of capillary blood for analysis in a point of care or medical lab.
26. The method of feature set 25, wherein the sample containment chamber is a standard medical analysis tube.
27. A method using the capillary blood sampling device of any one of feature sets 1 to 7, wherein, if the test subject tests positive for a pathogen, a treatment and/or quarantine protocol is initiated to ensure that a test subject having a positive test is handled in a manner to minimize the spread of the pathogen.
28. A method of feature set 25 wherein further the method includes the step of providing the result of the blood sample analysis and wherein the proof provided to the authorities to be used by the authorities to deliver an authorization for the user/patient for certain activities.
29. A method of feature set 25, wherein the method includes the step of using the result of the blood sample analysis and the proof provided to release the payment of a treatment.
30. A method of mass collection and analysis of an organic sample, the method consisting of collecting organic samples such as body fluid samples without the intervention of medically trained personnel, the method including at least the steps of:
a) providing the test subject with a sample device of any one of feature sets 1, 5-11, 13-24 having a unique identifier and instructions;
b) providing instructions, the instructions including instructions for placing the device in a refrigerator, to cool the device prior to and/or after taking a blood or other sample;
c) taking the sample and associating the sample device's unique identifier with the test subject;
d) transporting the sample to a collection site;
e) analyzing the sample to determine a pathogen and the test subject informed of the result.
31. A method of mass collection and analysis of an organic sample of feature set 30, the method including the steps of:
providing instructions, the instructions including instructions for to taking a blood or other sample;
taking the sample and associating the sample device's unique identifier with the test subject;
providing additional instructions, the instructions including optional instructions for placing the device in a refrigerator to cool the device.
   32. The method of the above feature set, including the additional step of, if the test subject tests positive for a pathogen indicating a treatment and/or quarantine protocol, to ensure that a test subject having a positive test is handled in a manner to minimize the spread of the pathogen.
   33. A method of mass collection and analysis of an organic sample, the method consisting of collecting organic samples such as body fluid samples without the intervention of medically trained personnel, the method including at least the steps of:
   a) providing the test subject with a sample device of any one of feature sets 1, 5-11, 13-24 having a unique identifier and instructions;
   b) providing the instructions including instructions for to taking a blood or other sample;
   c) taking the sample and associating the sample device's unique identifier with the test subject;
   d) providing further instructions, optionally, the instructions including instructions for placing the device in a refrigerator to cool the device, optionally before step c) when the sample is taken, and further optionally including instructions for sliding an insulating sleeve over a sample containment chamber in the device after the device has reached a temperature within an acceptable temperature range, thus prolonging the time which the sample can be safely stored during transport and before sample analysis of step f);
   e) transporting the sample to a collection site;
   f) analyzing the sample to determine a pathogen and the test subject informed of the result.
   34. A method of mass collection and analysis of an organic sample, the method consisting of collecting organic samples such as body fluid samples without the intervention of medically trained personnel, the method including at least the steps of:
   a) providing the test subject with a sample device of any one of feature sets 1, 5-11, 13-24 having a unique identifier and instructions;
   b) providing the instructions, optionally, the instructions including instructions for placing the device in a refrigerator, to optionally cool the device prior to and/or after taking a blood or other sample;
   c) optionally placing the sample device in a refrigerator for an instructed time and following the instructions optionally after step d) when the sample is taken, and further optionally including instructions for sliding an insulating sleeve over a sample containment chamber in the device after the device has reached a temperature within an acceptable temperature range, thus prolonging the time which the sample can be safely stored during transport and before sample analysis of step f);
   d) taking the sample and associating the sample device's unique identifier with the test subject;
   e) transporting the sample to a collection site;
   f) analyzing the sample to determine a pathogen and the test subject informed of the result.
   35. The method of the above feature set, including the further step of, if the test subject tests positive for a pathogen, initiating treatment and/or quarantine protocol to ensure that a test subject having a positive test is handled in a manner to minimize the spread of the pathogen, such as by following a quarantine protocol.
   36. A system able to verify the proper execution of self-administered medical processes such as blood sampling or injection, the system using one of the devices of any one of feature sets 1, 5-11, or 23-24 including a biometric scanner configured to recognize unique biometric characteristics of the user/patient such as face, scalp, eyes, fingerprints, and a video recorder for recording a video or time-lapse of the process execution.
   37. The system of feature set 36 in combination with an app running on a smartphone.
   38. The system of feature set 36 a device identification module adapted to, in real-time, recognize the device executing the blood sampling or injection process.
   39. The system of feature set 38 a device identification module adapted to, in real-time, analyze the execution of the process by the user/patient and/or by the sampling/injection device.
   40. The system of feature set 39 including a device identification module adapted to, in real-time, dispense audible and/or visual instructions for the user/patient to run the process correctly.
   41. The system of feature set 39 including a triggering module configured to wirelessly trigger parts or whole of the sampling or injection process.
   42. The system of any one of the preceding feature sets 36-41 including a connection mechanism adapted to provide appropriate connectivity to communicate in real-time with health authorities while the process is being run.
   43. The system of any one of the preceding feature sets 36-42 including a self-contained application running on a smartphone, including the storage of the process execution video or time-lapse in a secure manner in the smartphone for later usage as a proof
   44. The system of feature set 33 where the proof provided is adapted for use by the authorities to deliver an authorization for the user/patient for certain activities.
   45. The system of feature set 33 where the proof provided is adapted for use to release the payment of a treatment.
   46. A system for capillary blood sampling of any one of feature sets 36-45 includes a patient biometric based authentication system, a device authentication system and a disposable capillary blood sampling device providing a non-medically trained user with the ability to (a) sample a capillary blood, optionally auto-sampling; (b) optionally, using one or more droplet(s) of the sampled capillary blood, to immediately analyze blood; and (c) provide a standard medical analysis tube filled with a sample of capillary blood for analysis in a point of care or medical lab, the device including an interface for and a vacuum tube, the vacuum tube adapted to provide suction necessary to fill the vacuum tube with the blood.
   47. A capillary blood sampling system according to feature set 46, wherein the authentication system is adapted to provide real-time recognition of the user/patient and of the device executing the blood sampling process.
   48. A capillary blood sampling system according to feature set 46, wherein the authentication system includes storage means to store the sampling process execution video or time-lapse in a secure manner for later use as a proof.
   49. A method using the capillary blood sampling system of any one of the feature sets 36-48, wherein the result of the blood sample analysis and the proof provided are used by the authorities to deliver an authorization for the user/patient for certain activities.
   50. A method using the capillary blood sampling system of any one of the feature sets 36-48, wherein the result of the blood sample analysis and the proof provided are used to release the payment of a treatment.
   51. The disposable body fluid sampling device of feature set 1, wherein the device includes an adhesive integrated dressing adapted to (i) ensure the attachment of the blood sampling device to the patient's skin, (ii) ensure the air-tightness between the patient's skin and the blood sampling device during the sampling process, and (iii) the dressing of the wound after the sampling process.

Further, the invention should be considered as comprising all possible combinations of every feature described in the instant specification, appended claims, and/or drawing figures which may be considered new, inventive and industrially applicable.

It should be appreciated that the particular implementations shown and herein described are representative of the invention and its best mode and are not intended to limit the scope of the present invention in any way.

As will be appreciated by skilled artisans, the present invention may be embodied as a system, a device, or a method.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited, but may also include other elements described in the instant specification. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Additional features and functionality of the invention are described in the claims appended hereto. Such claims are hereby incorporated in their entirety by reference thereto in this specification and should be considered as part of the application as filed.

Multiple variations and modification are possible in the embodiments of the invention described here. For example, the cutting blades 1004 can take on any form, not limited to cutting blades 302, 5450, 5456, 3260, 3360, 3460 herein disclosed. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

1. A disposable body fluid sampling device providing a non-medically trained user with the functionalities of (a) sampling a body fluid, preferably blood, optionally auto-sampling; (b) optionally dispensing one or more droplet(s) of the sampled fluid for immediate analyses; and (c) providing a sample containment chamber filled with a sample of the body fluid for analysis in a medical lab, the device further including an interface for and a vacuum tube, the vacuum tube providing suction necessary to fill the vacuum tube with the blood.

2. The disposable body fluid sampling device of claim 1, wherein the vacuum tube is a standard medical analysis tube.

3. The disposable body fluid sampling device of claim 1 combined with an app running on a computer, the combination adapted for collecting a sample, wherein the computer is ecoded with instructions to execute a verification method comprising the steps of:
a) recognizing
unique biometric characteristics of the user/patient such as face, scalp, eyes, fingerprints,
physical devices adapted for the execution of the medical process such as cutting device, sampling device, vaccination device, sample container, vaccine container
b) saving a video of the user-patient and the device throughput the entire process together using a camera of the computer;
c) analyzing the video in real time and providing instructions to the user-patient for the correct execution of the medical process; and
d) storing the video in tamper-proof manner in the portable device or in a remotely accessible database through the portable device,
wherein at least one of the physical devices adapted for the execution of the medical process includes at least one visible feature that visibly signals the progress of the medical process.

4. (canceled)

5. A disposable body fluid sampling device of claim 1, having a sample containment chamber made of a material having a thermal inertia permitting the maintenance of sample temperature over a known period of time.

6. The disposable body fluid sampling device of claim 5, wherein the thermal inertia is selected to provide a known period of time of storage in an ambient environment sufficient to allow non-refrigerated transport to a collection point.

7. The device of the above claim wherein the known period of time is within a range of 1 hour to 2 hours under normal ambient conditions, and preferably within a range of 1 hour to 6 hours, and more preferably within a range of 1 hour to 8 hours.

8. The disposable body fluid sampling device of claim 5 including a thermally insulating sleeve configured to be manually or automatically triggered to slide over the sample container chamber.

9. The disposable body fluid sampling device of the above claim wherein the thermal inertia is selected to provide a known period of time sufficient to allow non-refrigerated transport to a collection point.

10. The disposable body fluid sampling device of the above claim wherein the known period of time is within a range of 1 hour to 2 hours under normal ambient conditions, and preferably within a range of 1 hour to 6 hours, and more preferably within a range of 1 hour to 8 hours.

11. (canceled)

12. A cutting blade made for making a laceration in the skin of a user/patient for the disposable body fluid sampling device of claim 1, wherein the cutting blade construction is made in one piece of material and provides the energy and the guiding for its movement.

13. An injection device and app combination for drug or vaccine injection which provides a non-medically trained user with the ability to perform a self-injection adapted to interact with the app connected, preferably in a wireless manner, to the Internet optionally via the Cloud, the app including means to allow for verification of the patient's ID and/or the particular device used, the combination including at least the following:
a) access to data storage adapted to store data of the physician or healthcare responsible (MED) for the vaccines/drug administration and his/her specific approval for the use of the vaccines and/or drug administration to the user/patient, wherein the combination is adapted to receive data related to the vaccine and/or drug entered by the user/patient, or by the HMO who received it previously;
b) optionally, a recording means adapted to record temperature of the vaccines and/or drug administration from shipment to substantially the time of injection as required;
c) patient safety means adapted to be activated in case of patient reaction
i) immediately at the time of injection (e.g. fainting); and/or
ii) after the vaccine, wherein, for aforementioned situation i), the combination according to the present invention is adapted to receive from the user/patient an input,
wherein further, the device is adapted to be shipped in thermal protection and the combination optionally including a temperature tracer or reagent paper, and a GPS tracer which tracks storage temperature during shipment.

14. The combination of claim 13, wherein the device is adapted to receive input by a user pushing a physical button or a button in the app, such to validate that the injection has been given, optionally, the device having means such as a flag adapted to indicate that the injection has been given.

15. The combination of the above claim, wherein, if there is no validation after a certain time after the patient has initiated the injection, the device is adapted to trigger an alarm to alert the MED, and, optionally, trigger an emergency procedure.

16. The combination of claim 13, wherein, for the aforementioned situation ii), the device of the combination is configured to allow the user/patient to confirm to the MED that he or she is fine.

17. The combination of claim 13, wherein the tracers are adapted to be returned to the vaccine and/or drug producer.

18. The combination of claim 13, wherein a timer is provided in the app which is configured to be switched on in the app when the user/patient takes the device out of its thermal protection, wherein warnings have to be given by the combination to the user/patient related with this event.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A system able to verify the proper execution of self-administered medical processes such as blood sampling or injection, the system including a biometric scanner configured to recognize unique biometric characteristics of the user/patient such as face, scalp, eyes, fingerprints, and a video recorder for recording a video or time-lapse of the process execution, wherein a device identification module is adapted to, in real time, recognize the device executing the blood sampling or injection process, wherein the device identification module is adapted to, in real-time, analyze the execution of the process by the user/patient and/or by the sampling/injection device, wherein the device identification module is adapted to, in real-time, dispense audible and/or visual instructions for the user/patient to run the process correctly, the system further including a connection mechanism adapted to provide appropriate connectivity to communicate in real-time with health authorities while the process is being run.

37. The system of claim 36 in combination with an app running on a smartphone.

38. (canceled)

39. (canceled)

40. The system of claim 37 including a device identification module adapted to, in real-time, dispense audible and/or visual instructions for the user/patient to run the process correctly including a connection mechanism adapted to provide appropriate connectivity to communicate in real-time with health authorities while the process is being run.

41. The system of claim 40 including a triggering module configured to wirelessly trigger parts or whole of the sampling or injection process.

42. (canceled)

43. The system of claim 36 including a self-contained application running on a smartphone, including the storage of the process execution video or time-lapse in a secure manner in the smartphone for later usage as a proof.

44-51. (canceled)
